(19) United States
(12) Patent Application Publication
(10) Pub. No.: US 2014/0010344 A1
(43) Pub. Date: Jan. 9, 2014

(54) MEDICAL IMAGE DISPLAY SYSTEM

(71) Applicants: Sumiya Nagatsuka, Hino-shi (JP); Junko Kiyohara, Hino-shi (JP); Shikou Kaneko, Tokorozawa-shi (JP); Kazuhiro Kido, Hino-shi (JP)

(72) Inventors: Sumiya Nagatsuka, Hino-shi (JP); Junko Kiyohara, Hino-shi (JP); Shikou Kaneko, Tokorozawa-shi (JP); Kazuhiro Kido, Hino-shi (JP)

(73) Assignee: KONICA MINOLTA, INC., Tokyo (JP)

(21) Appl. No.: 14/006,838

(22) PCT Filed: Mar. 22, 2012
(86) PCT No.: PCT/JP2012/057404

§ 371 Date: Sep. 23, 2013

(30) Foreign Application Priority Data
Mar. 23, 2011 (JP) 2011-063470

(51) Int. Cl. A61B 6/00

(52) U.S. Cl. CPC A61B 6/52; A61B 6/502
USPC 378/37; 378/62; 378/70

(57) ABSTRACT

A medical image display system is shown. The medical image display system includes, a fringe scanning type capturing apparatus or a Fourier transformation type capturing apparatus; an image processing section; a display section; and a control section. The capturing apparatus includes, an X-ray source; a first grating and a second grating; a subject table; and an X-ray detector. The image processing section generates a plurality of reconstructed images for diagnosis based on an image signal of a subject captured with the capturing apparatus. The display section displays at least two of the plurality of reconstructed images. The control section detects an abnormal candidate on each of the plurality of reconstructed images and controls display order of the plurality of reconstructed images displayed on the display section based on a result of detecting.

### TECHNICAL FIELD

The present invention relates to a medical image display system.

### BACKGROUND ART

Most medical X-ray images used in diagnosis are images by an absorption contrast method. According to the absorption contrast method, a contrast is formed by a difference of attenuation of X-ray intensity when X-rays pass through a subject. Alternatively, a phase contrast method is proposed, where contrast is obtained by phase variation of X-rays instead of absorption of X-rays. For example, phase contrast capturing is performed to obtain an X-ray image with high visibility by edge enhancement using refraction of X-rays in enlarged capturing (for example, see patent documents 1 and 2).

The absorption contrast method is effective for capturing a subject with large X-ray absorption, such as bones, etc. Alternatively, the phase contrast method can image tissue of breasts, articular cartilage, and soft tissue around joints, which have small X-ray absorption difference and which are difficult to be imaged by the absorption contrast method. Therefore, it is desired to apply the phase contrast method to X-ray image diagnosis.

As one method of phase contrast capturing, a Talbot interferometer using a Talbot effect is considered (for example, patent documents 3 to 5). The Talbot effect is an effect as follows, when coherent light passes through a first grating provided with slits at a certain cycle, the grating images are formed at a certain cycle in the travelling direction of light. The grating image is called a self image. The Talbot interferometer provides a second grating in a position where the self image is formed, and measures an interference fringe caused by slightly misaligning the second grating. Moire becomes unstable when something is placed in front of the second grating. Therefore, when X-ray capturing is performed by the Talbot interferometer, it is possible to obtain a reconstructed image of the subject by placing a subject in front of the first grating, emitting coherent X-rays, and calculating the obtained moire image.

Alternatively, a Talbot-Lau interferometer is proposed, which provides a multi-slit between the X-ray source and the first grating to increase the amount of emitted X-rays (for example, see patent document 6). A conventional Talbot-Lau interferometer captures a plurality of moire images at a certain cyclic interval while moving the first grating or the second grating (relatively moving both gratings), and the multi-slit is provided to increase the amount of X-rays.

Alternatively, the applicants of the present application filed a patent application of a system for a Talbot-Lau interferometer which moves the multi-slit in relation with the first grating and the second grating to enable scanning with high mechanical accuracy to obtain a high-definition image (see patent document 7). The applicants of the present application filed a patent application of a system for a Talbot-Lau interferometer which can obtain a high-definition image (see patent document 8).

As a method to create a reconstructed image from a moire image, in addition to creating the reconstructed image from a plurality of moire images of a certain cyclic interval obtained by the Talbot interferometer or the Talbot-Lau interferometer by a fringe scanning method as described above, there is known a method of creating a reconstructed image from one moire image using a Fourier transformation method (for example, see non-patent document 1). Although the space resolution of the reconstructed image obtained by the Fourier transformation method reduces compared to that of the fringe scanning method, unlike the fringe scanning method, a plurality of moire images are not necessary. Therefore, it is possible to shorten capturing time, and to reduce influence of body movement of the subject during capturing performed a plurality of times. Moreover, since there is no mechanic operation for scanning during capturing, there are no false images due to error of the sending mechanism of the grating or the multi-slit.

When a patient is monitored over time using the X-ray image, usually, capturing is performed in the same position under same capturing conditions as those of the past images of the patient, and the diagnosis image captured this time and the past images are both displayed tiled so that the physician can easily compare and interpret the images (for example, see patent document 9).

Moreover, when a certain lesion is diagnosed, for example, a tumor or calcification of the breast is diagnosed, in addition to the X-ray image of the patient to be diagnosed, a typical sample image of the suspected lesion, a teaching image, a normal image, etc. are displayed tiled to increase accuracy of diagnosis. Moreover, a result of an abnormal shadow candidate detected by CAD (Computer-Aided Diagnosis) is used as diagnosis assistance information (for example, see patent document 10). Further, both an X-ray image and an ultrasonic image are used for diagnosis (for example, see patent document 11).

### PRIOR ART DOCUMENT

### Patent Document

Patent Document 1: Japanese Unexamined Patent Application Publication No. 2007-268033
Patent Document 2: Japanese Unexamined Patent Application Publication No. 2008-18060
Patent Document 3: Japanese Unexamined Patent Application Publication No. S58-16216
Patent Document 4: WO 2004/058070
Patent Document 5: Japanese Unexamined Patent Application Publication No. 2007-203063
Patent Document 6: WO 2008/102898
Patent Document 7: WO 2011/033798
Patent Document 8: WO 2011/114845
Patent Document 9: Japanese Unexamined Patent Application Publication No. 2010-51523
Patent Document 10: Japanese Unexamined Patent Application Publication No. 2004-230001
Patent Document 11: Japanese Unexamined Patent Application Publication No. 2008-161283

### Non-Patent Document

Non-Patent Document 1: M. Takeda, H. Ina, and S. Kobayashi “Fourier-Transform Method of Fringe-Pattern Analysis for Computer-Based Topography and Interferometry” J. Opt. Soc. Am. 72,156 (1982)
Non-Patent Document 2: Tomoharu Yamada, Shunsuke Yokoseki, “Application Measurement Method of Moire Fringe and Interference Fringe”, Corona Publishing Co., Ltd., Dec. 10, 1996

### DISCLOSURE OF THE INVENTION

### Problems to be Solved by the Invention

When the result of the abnormal shadow candidate detected by the CAD is used as diagnosis assistance information, the result detected by the CAD is used as a second opinion for a result interpreted by a physician based on the X-ray image (absorption image). Depending on how the algorithm for detecting the abnormal shadow candidate with the CAD is applied (setting a threshold for judging whether it is an abnormal shadow candidate relatively high to detect a candidate which is at least clearly true positive or setting the threshold relatively low to prevent candidates from not being detected), the time used for interpretation of the image greatly differs, the degree of fatigue of the physician differs, and the diagnosis accuracy is not stable. When both the X-ray breast image and the ultrasonic image are used for diagnosis, capturing of two systems need to be performed, and this puts a heavy burden on the physician and the examination subject (regarding both number of steps and fee).

An object of the present invention is to effectively use a reconstructed image created from a moire image generated by a Talbot interferometer or a Talbot-Lau interferometer to enable early diagnosis and to enhance diagnosis accuracy.

### Means for Solving the Problem

In order to achieve the above object, according to one aspect of the present invention, there is provided a medical image display system comprising:

a fringe scanning type capturing apparatus or a Fourier transformation type capturing apparatus including,
an X-ray source which emits an X-ray;
a first grating and a second grating in which a plurality of slits are provided in an array in a direction orthogonal to an emitting axis direction of the X-ray;
a subject table; and
an X-ray detector including a conversion element provided two-dimensionally to generate an electric signal according to the emitted X-ray so that the X-ray detector reads the electric signal generated by the conversion element as an image signal;

an image processing section which generates at least two among an X-ray absorption image, a differential phase image, and a small angle scattering image of a subject based on the image signal of a subject captured with either of the capturing apparatuses;

a display section which displays the images generated by the image processing section; and

a control section which controls display of the images generated by the image processing section displayed on the display section.

Preferably, the control section controls display on the display section to sequentially switch display between at least two images generated by the image processing section each time a predetermined amount of time passes.

Preferably, the fringe scanning type capturing apparatus is a Talbot-Lau interferometer which includes a multi-slit provided near the X-ray source and which moves the multi-slit relatively with respect to the first grating and the second grating.

### Advantageous Effect of the Invention

According to the present invention, it is possible to effectively use a reconstructed image created from a moire image generated by a Talbot interferometer or a Talbot-Lau interferometer to enable early diagnosis and to enhance diagnosis accuracy.

### BRIEF DESCRIPTION OF THE DRAWINGS

FIG. 1 is a medical image display system (including a side view image of an X-ray capturing apparatus) of the present embodiment;

FIG. 2A is a planar view of a multi-slit;

FIG. 2B is a planar view and a side view of a state of a holder holding the multi-slit;

FIG. 3 is a planar view and a side view of a multi-slit rotating section;

FIG. 4A is a planar view of a subject holder;

FIG. 4B is a side view of a subject holder;

FIG. 5 is a planar view of a grating rotating section;

FIG. 6 is a planar view and a side view of a grating rotating section in a state with a first grating and a second grating attached;

FIG. 7A is a planar view showing an enlarged holding portion of the grating rotating section in the holding section of FIG. 1;

FIG. 7B is a cross-sectional view along line E-E′ as shown in FIG. 7A;

FIG. 7C is a diagram showing a state where the holding section holds the grating rotating section;

FIG. 7D is a cross-sectional view showing a rotating tray which can rotate an X-ray detector together with the first grating and the second grating;

FIG. 8 is a block diagram showing a functional configuration of a main section;

FIG. 9 is a block diagram showing a functional configuration of a controller;

FIG. 10 is a diagram describing a principle of a Talbot interferometer;

FIG. 11 is a flowchart showing a capturing control processing by a control section of the X-ray capturing apparatus;

FIG. 12 is a flowchart showing a first capturing mode processing performed in step S2 shown in FIG. 11;

FIG. 13 is a flowchart showing a second capturing mode processing performed in step S3 shown in FIG. 11;

FIG. 14 is a flowchart showing a reconstructed image creating/displaying processing;

FIG. 15 is a diagram for explaining an X-ray intensity variation correction among a plurality of moire images;

FIG. 16 is a diagram showing a moire image obtained by capturing of 5 steps;

FIG. 17 is a graph showing an X-ray relative intensity of a target pixel of a moire image of each step;

FIG. 18A is a diagram showing an example of an absorption image;

FIG. 18B is a diagram showing an example of a differential phase image;

FIG. 18C is a diagram showing an example of a small angle scattering image;

FIG. 19 is a flowchart showing a reconstructed image creating/displaying processing by a Fourier transformation method performed by the control section of the controller;

FIG. 20A is a diagram showing an example of a moire image with a subject captured in a second capturing mode;

FIG. 20B is a diagram showing a result of two-dimensional Fourier transformation of the moire image shown in FIG. 20A;

FIG. 21A is a diagram showing an example of a moire image without the subject captured in the second capturing mode;

FIG. 21B is a diagram showing a result of two-dimensional Fourier transformation of the moire image shown in FIG. 21A;

FIG. 22 is a diagram showing a grating direction when a slit direction of grating is provided vertically, an interference fringe captured in the first capturing mode, an interference fringe captured in the second capturing mode, and a result of Fourier transformation of the interference fringe captured in the second capturing mode;

FIG. 23 is a diagram showing a grating direction when a slit direction of grating is provided horizontally, an interference fringe captured in the first capturing mode, an interference fringe captured in the second capturing mode, and a result of Fourier transformation of the interference fringe captured in the second capturing mode;

FIG. 24 is a diagram showing a grating direction when a slit direction of grating is provided slanted, an interference fringe captured in the first capturing mode, an interference fringe captured in the second capturing mode, and a result of Fourier transformation of the interference fringe captured in the second capturing mode;

FIG. 25 is a diagram showing an example of a zero order component obtained by Fourier transformation cut out with a Hanning window;

FIG. 26 is a diagram showing an example of a first order component obtained by a Fourier transformation shifted in an amount of a carrier frequency and cut out with a Hanning window;

FIG. 27 is a diagram showing an example of a window of an improved Fourier transformation;

FIG. 28A is a diagram showing an example of a reconstructed image of a subject obtained by a fringe scanning method;

FIG. 28B is a diagram showing an example of a reconstructed image obtained by an improved Fourier transformation method;

FIG. 28C is a diagram showing an example of a reconstructed image obtained by a conventional Fourier transformation method;

FIG. 29 is a diagram showing an example of a display of a reconstructed image of a first embodiment;

FIG. 30 is a diagram showing an example of a display of a reconstructed image of a second embodiment;

FIG. 31 is a diagram showing another example of a display of a reconstructed image of a second embodiment;

FIG. 32 is a diagram for describing a relation between an abnormal shadow candidate and a threshold;

FIG. 33A is a diagram showing an example of a diagnosis screen where an absorption image, a small angle scattering image, a differential phase image, and a switching display image are positioned tiled;

FIG. 33B is a diagram showing another example of a diagnosis screen where an absorption image, a small angle scattering image, a differential phase image, and a switching display image are positioned tiled; and

FIG. 34 is a diagram showing an example of a diagnosis screen where an absorption image and a switching display image are positioned tiled.

### EMBODIMENT FOR CARRYING OUT THE INVENTION

### First Embodiment

Below, the first embodiment of the present invention is described with reference to the drawings.

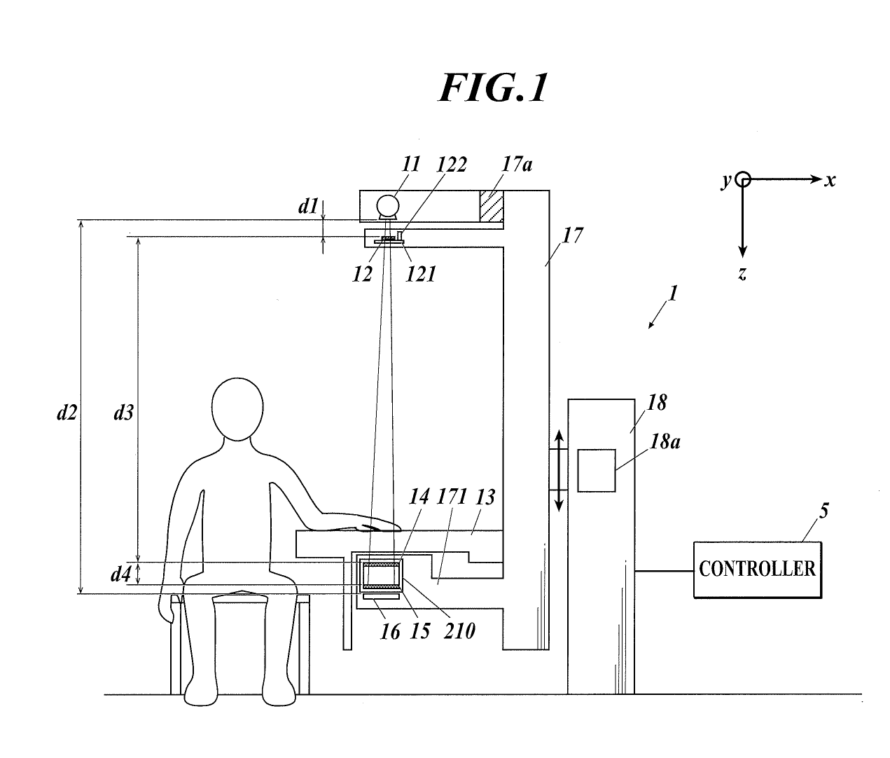

FIG. 1 shows a medical image display system of a first embodiment. The medical image display system includes an X-ray capturing apparatus 1 and a controller 5. The X-ray capturing apparatus 1 is an apparatus including a first capturing mode to function as a fringe scanning type capturing apparatus and a second capturing mode to function as a Fourier transformation type capturing apparatus. The fringe scanning type capturing apparatus performs capturing of a plurality of steps with a Talbot-Lau interferometer for a reconstructed image by a fringe scanning method to generate a plurality of moire images. The Fourier transformation type capturing apparatus performs capturing in one or two directions for a reconstructed image of a Fourier transformation method to generate one or two moire images.

The present embodiment describes an example where the configuration of the X-ray capturing apparatus 1 is described as an apparatus which captures hands and fingers as the subject. However, the present embodiment is not limited to the above.

As shown in FIG. 1, the X-ray capturing apparatus 1 includes an X-ray source 11, a multi-slit 12, a subject table 13, a first grating 14, a second grating 15, an X-ray detector 16, a holding section 17, a main section 18, and the like. The X-ray capturing apparatus 1 is a vertical type and the X-ray source 11, the multi-slit 12, the subject table 13, the first grating 14, the second grating 15, and the X-ray detector 16 are provided in this order in a z-direction which is the direction of gravity. The distance between a focus of the X-ray source 11 and the multi-slit 12 is shown by d1 (mm), the distance between the focus of the X-ray source 11 and the X-ray detector 16 is shown by d2 (mm), the distance between the multi-slit 12 and the first grating 14 is shown by d3 (mm), and the distance between the first grating 14 and the second grating 15 is shown by d4 (mm).

Preferably, the distance d1 is 5 to 500 (mm), and even more preferably, the above distance is 5 to 300 (mm).

Since the height of the capturing room of the radiology department is usually about 3 (m) or less, it is preferable that the distance d2 is at least 3000 (mm) or less. Within the above, preferably, the distance d2 is 400 to 2500 (mm), and even more preferably, the above distance is 500 to 2000 (mm).

Preferably, the distance between the focus of the X-ray source 11 and the first grating 14 (d1+d3) is 300 to 2800 (mm), and even more preferably, the above distance is 400 to 1800 (mm).

Preferably, the distance between the focus of the X-ray source 11 and the second grating 15 (d1+d3+d4) is 400 to 3000 (mm), and even more preferably, the above distance is 500 to 2000 (mm).

Each of the above distances can be set by calculating a suitable distance where a grating image (self image) by the first grating 14 overlaps on the second grating 15, from the wavelength of the X-ray emitted from the X-ray source 11.

The X-ray source 11, the multi-slit 12, the subject table 13, the first grating 14, the second grating 15, and the X-ray detector 16 are held together by the same holding section 17, and the relation of the position in the z-direction is fixed. The holding section 17 is formed as a C-shaped arm and is attached to the main section 18 to be able to move (rise and fall) in a z-direction with the driving section 18a provided in the main section 18.

The X-ray source 11 is held through a buffering member 17a. The buffering member 17a can be any material as long as the material can absorb shock or vibration, and as an example of such material there is elastomer, etc. Since the X-ray source 11 is heated by emitting the X-ray, it is preferable that the buffering member 17a on the X-ray source 11 side is also a heat insulating material.

The X-ray source 11 includes an X-ray tube, and the X-ray tube generates the X-ray to emit the X-ray in the z-direction (gravity direction). As the X-ray tube, for example, it is possible to use a Coolidge X-ray tube or a rotating anode X-ray tube typically used widely in the field of medicine. As the anode, it is possible to use tungsten or molybdenum.

Preferably, the diameter of the focus of the X-ray is 0.03 to 3 (mm) and even more preferably, 0.1 to 1 (mm).

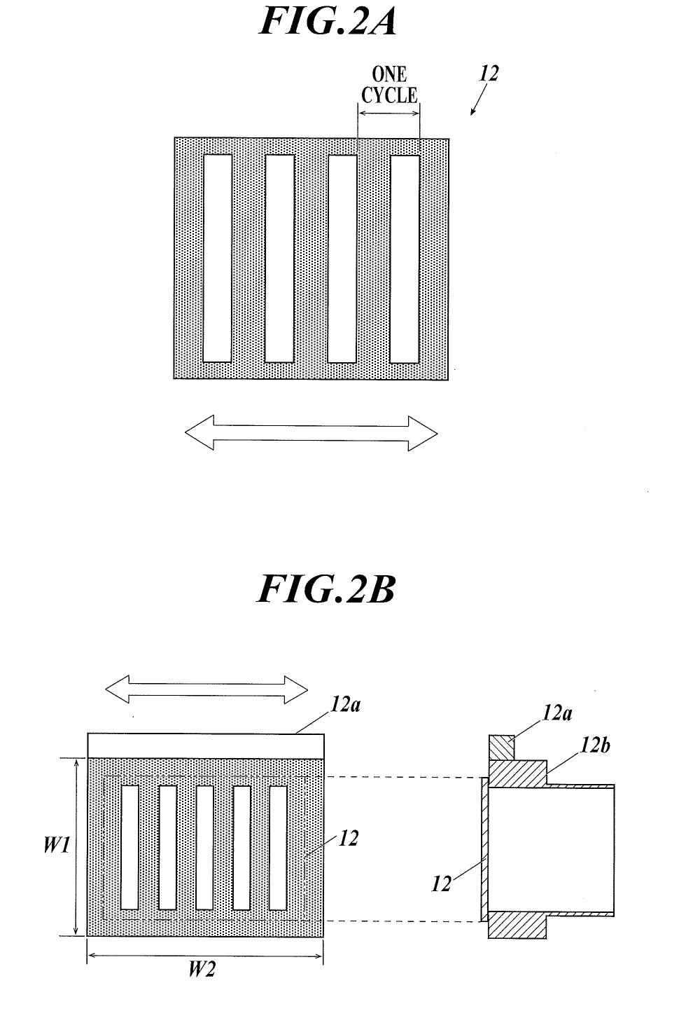

The multi-slit 12 is a diffraction grating, and as shown in FIG. 2A, a plurality of slits are provided in an array in a predetermined interval. The plurality of slits are provided in an array in a direction (shown with a white arrow in FIG. 2A) orthogonal to a direction (z-direction of FIG. 1) of the emitting axis of the X-ray. The multi-slit 12 is formed with a material with large strength of screening X-rays, in other words, material with a high absorption rate of X-rays, such as tungsten, lead, or gold on a substrate including material with a low absorption rate of X-rays, such as silicon or glass. For example, the resist layer is masked in a slit shape by photolithography, and UV is emitted so that the pattern of the slits is transferred on the resist layer. The slit configuration with the same shape as the pattern is obtained by exposure and metal is embedded between the slit configuration by electrocasting to form the multi-slit 12.

The slit cycle of the multi-slit 12 is 1 to 60 (μm). Regarding the slit cycle, the distance between adjacent slits as shown in FIG. 2A is considered to be one cycle. The width of the slit (length of the slit array direction of each slit) is the length 1 to 60(%) of the slit cycle, and is preferably, 10 to 40(%). The height of the slit (height in the z-direction) is 1 to 500 (μm), and is preferably 1 to 150 (μm).

When the slit cycle of the multi-slit 12 is w₀ (μm), and the slit cycle of the first grating 14 is w₁ (μm), the slit cycle w₀ can be obtained by the following formula.

w₀=w₁·(d3+d4)/d4

By determining the cycle w₀ to satisfy the above formula, the self image formed by the X-ray passing through each slit of the multi-slit 12 and the first grating 14 can each overlap on the second grating 15, and is able to become a state known as the focused state.

As shown in FIG. 2B, the multi-slit 12 is held by a holder 12 by including a rack 12a. The rack 12a is provided in a slit array direction of the multi-slit 12. The rack 12a is engaged with a pinion 122c of the later described driving section 122. The rack 12a is provided to move the multi-slit 12 held by the holder 12b in the slit array direction according to a rotation (phase angle) of the pinion 122.

According to the present embodiment, the multi-slit rotating section 121 and the driving section 122 are provided in the X-ray capturing apparatus 1. The multi-slit rotating section 121 is a mechanism to rotate the multi-slit 12 around the emitting axis of the X-ray according to rotation of the first grating 14 and the second grating 15 around the emitting axis of the X-ray. The driving section 122 is a mechanism to move the multi-slit 12 in the slit array direction to capture the plurality of moire images.

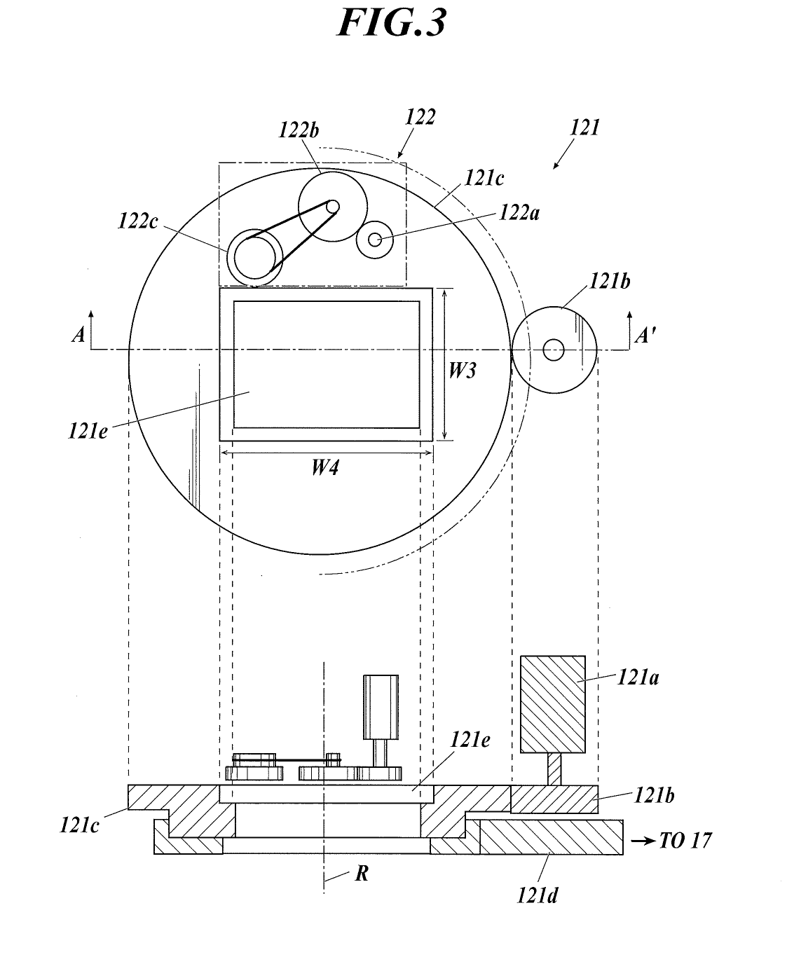

FIG. 3 shows a planar view of the multi-slit rotating section 121 and the driving section 122, and a cross-sectional view along line A-A′.

As shown in FIG. 3, the multi-slit rotating section 121 includes a motor section 121a, a gear section 121b, a gear section 121c, a supporting section 121d, and the like. The motor section 121a, the gear section 121b, and the gear section 121c are held by the holding section 17 through the supporting section 121d.

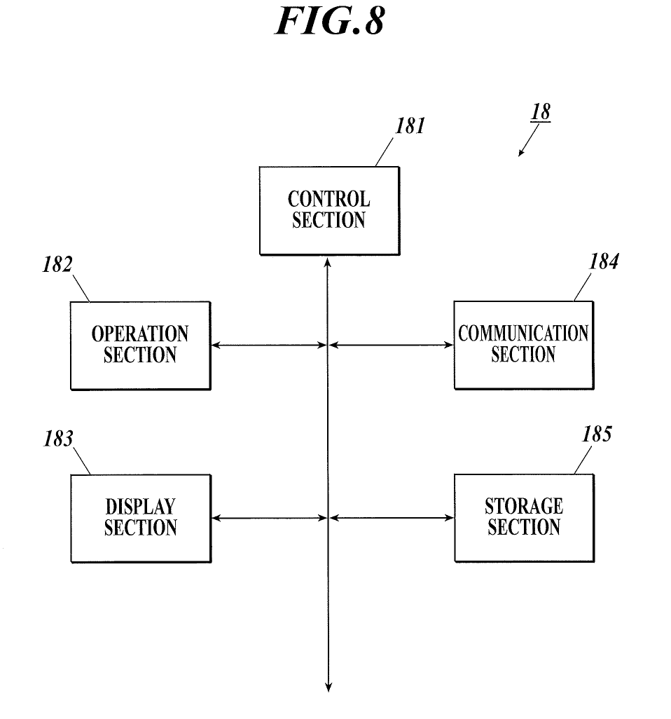

The motor section 121a is a pulse motor which can be switched to micro-step drive, and is driven according to control from the control section 181 (see FIG. 8) to rotate the gear section 121c around an X-ray emitting axis (shown with an alternate long and short dashed line R in FIG. 3) through the gear section 121b. The gear section 121c includes an opening section 121e to attach the multi-slit 12 held by the holder 12b. The gear section 121c is rotated to rotate the multi-slit 12 attached to the opening section 121e around the X-ray emitting axis so as to be able to change the slit array direction of the multi-slit 12. In the capturing, if the multi-slit 12 is able to rotate about 0° to 90°, this is enough. Therefore, the gear section 121c does not need to be provided around the entire circumference, and it is enough if the gear section 121c is able to rotate in a range shown with alternate long and two short dashed lines shown in FIG. 3 (90° each in the forward and reverse rotating direction).

The opening section 121e is a shape and size so that the multi-slit 12 held by the holder 12b can be fitted from the upper portion. Here, the size w4 of the opening section 121e in the slit array direction is slightly larger than the size W2 of the holder 12b in the slit array direction, and it is possible to slide the multi-slit 12 in the slit array direction. The size w3 of the opening section 121e in a direction orthogonal to the slit array direction is a measure which can accurately fit with the size W1 of the holder 12b in a direction orthogonal to the slit array direction. When the holder 12b is attached to the opening section 121e, a rack 12a provided on the holder 12b is positioned outside the opening section 121e so that it is possible to be engaged with a later described pinion 122c.

The driving section 122 includes an accurate reducer, etc. which moves the multi-slit 12 in a unit of 0.1 μm to several tens μm according to the multi-slit cycle in the slit array direction. As shown in FIG. 3, for example, the driving section 122 includes a motor section 122a, a gear section 122b, a pinion 122c, and the like. The driving section 122 is fixed to the gear section 121c of the multi-slit rotating section 121 by a letter L type plate not shown. With this, the multi-slit 12 and the driving section 122 can be rotated together.

For example, the motor section 122a is driven according to control from the control section 181, and rotates the pinion 122c through the gear section 122b. The pinion 122c is engaged to the rack 12a of the multi-slit 12 and rotates to move the multi-slit 12 in the slit array direction.

Returning to FIG. 1, the subject table 13 is a table for placing the hand and the finger which is the subject. It is preferable that the subject table 13 is provided in a height where the elbow of the patient can be placed. Since it is possible to place the elbow of the patient, the patient is positioned in a relaxed posture. Therefore, it is possible to reduce movement of the fingertip in the site to be captured during the capturing which continues for a relatively long period of time.

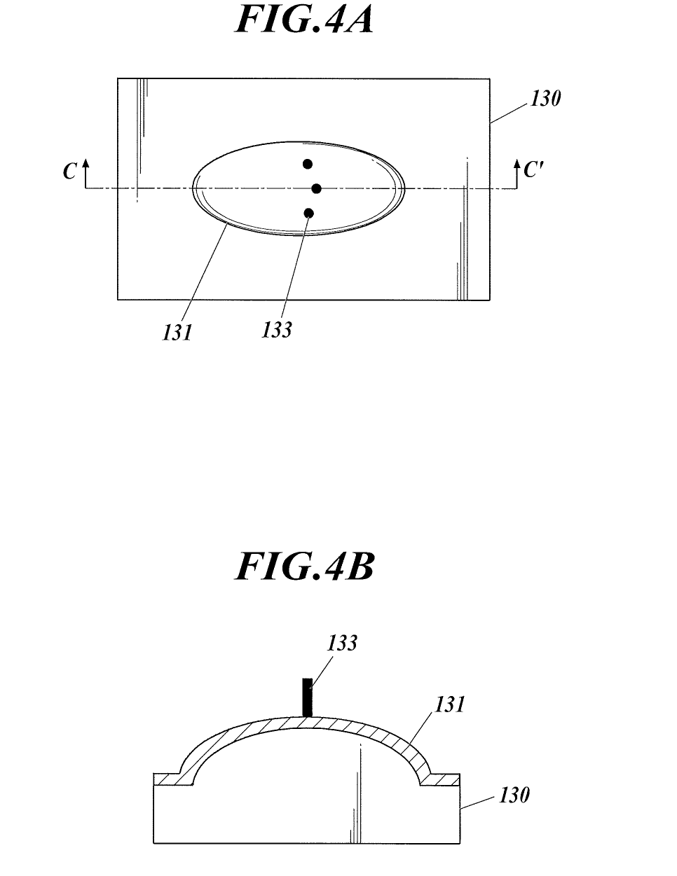

A subject holder 130 is provided on the subject table 13 to fix the subject. The subject holder 130 can be attached and detached according to the subject. As shown in FIG. 4A, the subject holder 130 is a plate member attached with an ellipse shaped 131 similar to a mouse which can be easily grabbed by a palm of a hand. As shown in FIG. 4B, when the cross section of the ellipse shaped 131 is viewed from the side, the cross section is a gentle convex curved surface in a size of the palm of the hand. The patient can grab the ellipse shaped 131 with the palm of the hand to maintain a state which is not tiresome for the subject and to reduce movement of the subject in a downward direction.

If the subject holder 130 has a shape or thickness with an uneven X-ray complex refraction index in some places, variation occurs in the X-ray amount which reaches the X-ray detector 16 due to the uneven X-ray complex refraction index of the subject holder 130.

Preferably, an interdigital spacer 133 is provided on the subject holder 130 to further stabilize the posture of the subject. Since the size of the hand and the space between the fingers is different for each patient, preferably, the subject holder 130 is made to fit the shape of the palm of the hand of each patient, and the subject holder 130 for a certain patient is attached to the subject table 13 with a magnet, etc., when the patient is captured. The weight from the arm to the wrist is supported by the subject table 13, and the subject holder 130 only needs to be able to withstand weight of the fingertip portion and the force pressed from above by the patient. Therefore, it is possible to employ plastic molding which is cheap in cost and which can be manufactured in large quantities.

Returning to FIG. 1, similar to the multi-slit 12, the first grating 14 is a diffraction grating provided with a plurality of slits in an array in the direction orthogonal to the z-direction which is the X-ray emitting axis direction. Similar to the multi-slit 12, the first grating 14 can be formed by photolithography using UV or the grating configuration can be formed from only silicon by performing deep evacuating processing with fine thin lines on a silicon substrate by an ICP method. The slit cycle of the first grating 14 is 1 to 20 (μm). The width of the slit is 20 to 70(%) of the slit cycle, and preferably, 35 to 60(%). The height of the slit is 1 to 100 (μm).

When a phase type is used as the first grating 14, the height (height in the z-direction) of the slit is to be the height where the phase difference between two types of material forming the slit cycle, in other words, the phase difference between the material of the X-ray passing section and the material of the X-ray screening section is π/8 to 15×π/8. Preferably, the height is where the phase difference is π/4 to 3×π/4. When the absorption type is used as the first grating 14, the height of the slit is the height where the X-rays are sufficiently absorbed by the X-ray screening section.

When the first grating 14 is the phase type, the distance d4 between the first grating 14 and the second grating 15 needs to substantially satisfy the following condition.

d4=(m+½)·w₁²/λ

Here, m is an integer and λ is a wavelength of the X-ray.

The above condition is for an example where the first grating 14 is a π/2 type grating, in other words, the phase difference between the materials of the X-ray screening section and the X-ray passing section of the first grating is near π/2. However, the it type can be used for the first grating 14 and the condition can be calculated according to the type of grating used.

Similar to the multi-slit 12, the second grating 15 is a diffraction grating provided with a plurality of slits in an array in a direction orthogonal to the z-direction which is the X-ray emitting axis direction. The second grating 15 can also be formed by photolithography. The slit cycle of the second grating 15 is 1 to 20 (μm). The width of the slit is 30 to 70(%) of the slit cycle, and preferably, 35 to 60(%). The height of the slit is 1 to 100 (μm).

According to the present embodiment, the first grating 14 and the second grating 15 each have a grating surface which is perpendicular to the z-direction (parallel in the x-y plane). The slit array direction of the first grating 14 and the slit array direction of the second grating 15 are positioned tilted a predetermined angle in the x-y plane, however, both directions can be positioned parallel. According to the present embodiment, the first grating 14 and the second grating 15 are disk shaped.

The multi-slit 12, the first grating 14, and the second grating 15 can be configured as described below.

The focus diameter of the X-ray tube of the X-ray source; 300 (μm), tube voltage: 40 (kVp), added filter: aluminum 1.6 (mm)

distance d1 from focus of X-ray source 11 to the multi-slit 12: 240 (mm)

distance d3 from the multi-slit 12 to the first grating 14: 1110 (mm)

distance d3+d4 from the multi-slit 12 to the second grating 15: 1370 (mm)

size of the multi-slit 12: 10 (mm for each of the four sides), slit cycle: 22.8 (μm)

size of the first grating 14: 50 (mm for each of the four sides), slit cycle: 4.3 (μm)

size of the second grating 15: 50 (mm for each of the four sides), slit cycle: 5.3 (μm)

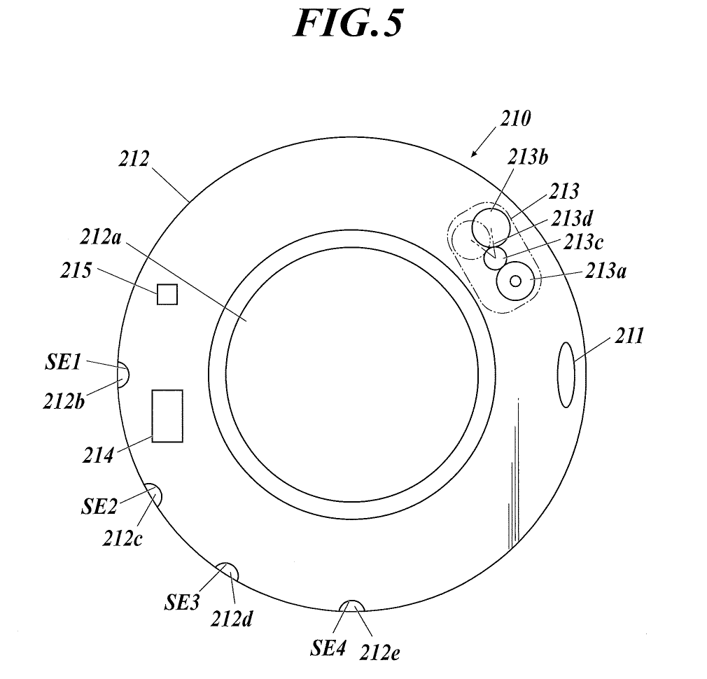

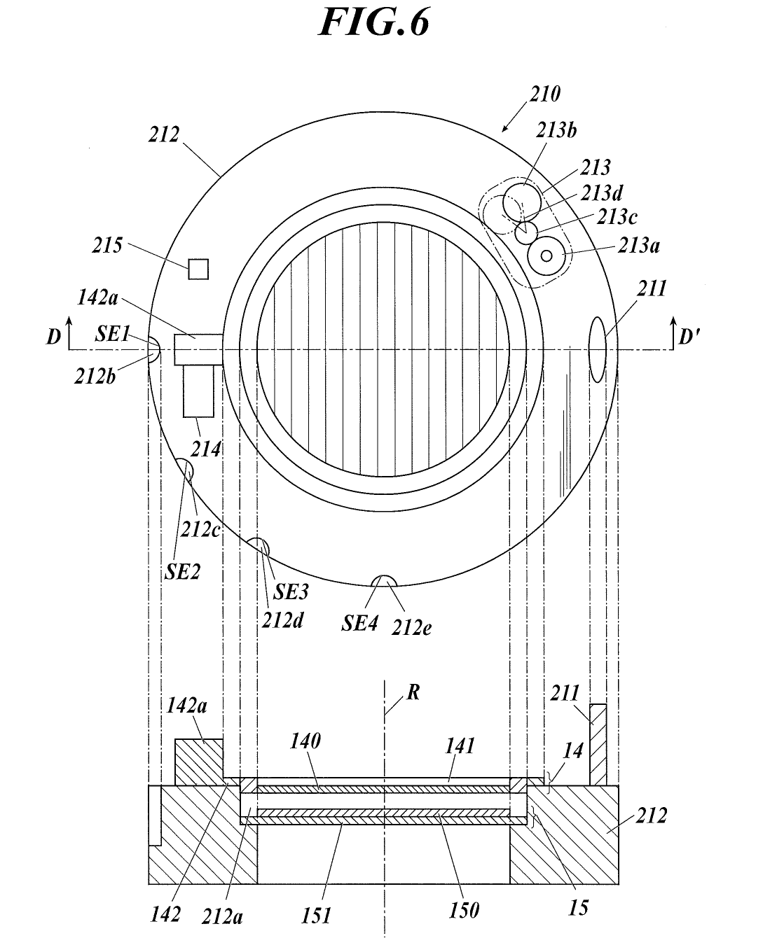

According to the present embodiment, the first grating 14 and the second grating 15 are attached to the grating rotating section 210. FIG. 5 shows a planar view of the grating rotating section 210. FIG. 6 shows a planar view of the grating rotating section 210 in a state with the first grating 14 and the second grating 15 attached, and a cross-sectional view along line D-D′.

As shown in FIG. 5, the grating rotating section 210 includes a handle 211, a relative angle adjusting section 213, a stopper 214, and the like on the rotating tray 212.

The rotating tray 212 includes an opening section 212a to hold the first grating 14 and the second grating 15.

Here, according to the present embodiment, the first grating 14 includes a circular grating section 140 with a plurality of slits in an array, a first holder section 141 and a second holder section 142 to attach the grating section 140 to the opening section 212a (see FIG. 6). The first holder section 141 is a member attached to the outer circumference of the grating section 140 with substantially the same radius (radius of the outer circumference) as the opening section 212a and fits with the opening section 212a when the first grating 14 is attached. The second holder section 142 is a member attached on the outer side than the first holder section 141a with a radius (radius of the outer circumference) rather larger than the opening section 212a. A portion of the outer circumference of the second holder section 142 is processed as a gear. A protrusion section 142a is provided in a predetermined position of the outer circumference of the second holder section 142.

The second grating 15 includes a circular grating section 150 with a plurality of slits in an array, and a holder section 151 to attach the grating section 150 to the opening section 212a. The holder section 151 is a disk shaped member with a radius substantially the same as the radius of the opening section 212a. The grating section 150 is held on the upper surface of the center section of the holder section 151 (see FIG. 6).

When the first grating 14 and the second grating 15 are attached to the rotating tray 212, first, the second grating 15 is fitted to the bottom surface of the opening section 212a. Next, the first grating 14 is fitted in the opening section 212a from above the second grating 15. With this, the first grating 14 and the second grating 15 are held in the rotating tray 212 in the state as shown in FIG. 6.

The relative angle between each slit direction of the first grating 14 and the second grating 15 held by the opening section 212a is adjusted by the relative angle adjusting section 213 according to the capturing mode.

Here, the X-ray capturing apparatus 1 includes a first capturing mode which performs capturing with a plurality of steps for a reconstructed image using a fringe scanning method, and a second capturing mode which performs capturing in 1 or 2 directions for a reconstructed image using a Fourier transformation method. The relative angle between the slit direction of the first grating 14 and the slit direction of the second grating 15 demanded in capturing for the fringe scanning method depends on the cycle of the second grating, the image size and the number of fringes. According to the fringe scanning method, it is known that as the number of interference fringes in the moire image becomes smaller or as the interference fringes become clearer, the reconstructed image created based on the moire image becomes clearer (see non-patent document 2). Therefore, assuming that the cycle of the second grating is 5.3 μm, and the number of interference fringes is about 0 to 3 in an image of 60 mm for each side, the relative angle needs to be 0 degrees to ±0.015 degrees. Alternatively, the relative angle between the slit direction of the first grating 14 and the slit direction of the second grating 15 demanded in capturing for the Fourier transformation method depends on the pixel pitch and the spatial resolution of the X-ray detector 16. Assuming a typically used detector (spatial resolution of 30 μm to 20 μm), the relative angle needs to be 0.4 degrees to 3 degrees. Therefore, in order to capture switching between the first capturing mode and the second capturing mode, the relative angle between the first grating and the second grating needs to be adjusted according to the capturing mode. However, for example, in the above configuration, a difference of 0.005 degrees of the angle in the fringe scanning method corresponds to one cycle of the fringe. Since adjustment with an accuracy in the unit of milli-degrees is demanded in order to maintain a state where the fringes are always spread in the fringe scanning method, it is difficult to adjust the relative angle between the slit directions of the first grating 14 and the second grating 15 manually.

Therefore, in the X-ray capturing apparatus 1, by using the relative angle adjusting section 213, it is possible to automatically adjust the relative angle between the first grating 14 and the second grating 15 according to the capturing mode set by the operation section 182.

As shown in FIG. 5 and FIG. 6, the relative angle adjusting section 213 includes a motor section 213a, a first gear 213b, a second gear 213c, and a lever 213d. The motor section 213a is engaged with the second gear 213c and rotates the second gear 213c according to control from the control section 181. The center of the second gear 213c is connected with the center of the first gear 213b through the lever 213d, and the circumference is engaged with the first gear 213b. When the second gear 213c rotates according to drive of the motor section 213a, the first gear 213b rotates along the perimeter of the second gear 213c with the center of the second gear 213c as the rotating axis to engage with the gear portion of the second holder section 142 of the first grating 14. With this, it is possible to rotate the first grating 14 around the X-ray emitting axis without rotating the second grating 15.

According to the present embodiment, when shipped from the factory, the position of the stopper 214 and the relative angle between the first grating 14 and the second grating 15 are adjusted in advance to be attached to the opening section 212a so that the relative angle of the slit directions between the first grating 14 and the second grating 15 becomes a suitable relative angle in the first capturing mode (capturing mode for fringe scanning method) when the protrusion section 142a of the second holder section 142 strikes the stopper (convex protrusion) 214 provided on the rotating tray 212. When the second capturing mode (capturing mode for Fourier transformation) is set, the motor section 213a employing a pulse motor of the relative angle adjusting section 213 is driven (energizing control) by the control section 181 so that the relative angle between the first grating 14 and the second grating 15 becomes suitable for the second capturing mode. With this, the first gear 213b rotates through the second gear 213c to engage with the gear portion of the second holder section 142, and the second holder section 142 rotates so that the relative angle of the slit directions between the first grating 14 and the second grating 15 becomes suitable for the second capturing mode. Then, the energized state of the pulse motor is changed, and the phase of the second holder section 142 can be maintained by setting an energized state to a degree where a motor self maintaining strength (excitation force) is exerted to overcome a later described spring force (not more than 50% of the rated current in driving, etc.).

Since the rotating angle is about 1 degree which is small, it is preferable that first, the pulse motor of the motor section 213a rotates the second holder section 142 counterclockwise so that the protrusion section 142a reaches a reference position 215, and when a sensor not shown detects that the protrusion section 142a reaches the reference position 215, the rotating direction of the second holder section 142 is switched to clockwise to rotate the second holder section 142 by micro-step driving.

The second holder section 142 is biased by a spring not shown, and when the engagement between the first gear 213b and the second holder section 142 is released by the driving of the motor section 213a, the protrusion section 142a returns to the position of the stopper 214 by the bias force of the spring. In other words, the first grating 13 and the second grating 15 returns to the relative angle suitable for the first capturing mode.

As described above, the relative angle between the first grating 14 and the second grating 15 is adjusted to an angle appropriate for the capturing mode.

The first grating 14 and the second grating 15 with the relative angle adjusted can be rotated as one with respect to the subject around the X-ray emitting axis (shown with a dotted line R in FIG. 6) by the grating rotating section 210.

Here, there is a characteristic that a configuration which stretches in a line parallel to the slit direction of the first grating 14 and the second grating 15 cannot be clearly captured by the Talbot interferometer and the Talbot-Lau interferometer using the one-dimensional grating (slit). Therefore, the angle of the slit directions between the first grating 14 and the second grating 15 needs to be adjusted according to the positioning direction of the target configuration of the subject. According to the mechanism below, the grating rotating section 210 is able to rotate as one the first grating 14 and the second grating 15 with the relative angle maintained around the X-ray emitting axis and is able to adjust the angle of the slit directions of the first grating 14 and the second grating 15 with respect to the positioning direction of the target configuration of the subject.

As described above, the rotating tray 212 is provided with a handle 211. The handle 211 is a protrusion for an operator such as a capturing technician, etc. to rotate the rotating tray 212 manually with the X-ray emitting axis (shown with a dotted line R in FIG. 6) as the axis. The rotating tray 212 includes depressed sections 212b to 212e for fixing a rotating angle of the rotating tray 212. The depressed sections 212b to 212e are provided in a position with a predetermined rotating angle (here, 0°, 30°, 60°, 90°) from a position set in advance as 0° (here, the position where the depressed section 212b is opposite of the ball of the tray fixing member 171b is to be the position of 0°). Each of the depressed sections 212b to 212e are provided with angle detecting sensors SE1 to SE4, which detect engagement with the tray fixing member 171b to output the detecting signal to the control section 181.

As described above, since the rotating tray 212 manually rotates, there is no need to provide an electric cord, etc. to rotate the first grating 14 and the second grating 15 as one in a range which comes into contact with the patient. Therefore, safety can be secured.

According to the present embodiment, each of the positions (angle) of the first grating 14 and the second grating 15 when the rotating tray 212 is set to 0° is to be the home position. When the first grating 14 and the second grating 15 are in the home position, the position (angle) where the slit direction of the first grating 14 and the slit direction of the multi-slit 12 are parallel is to be the home position of the multi-slit 12.

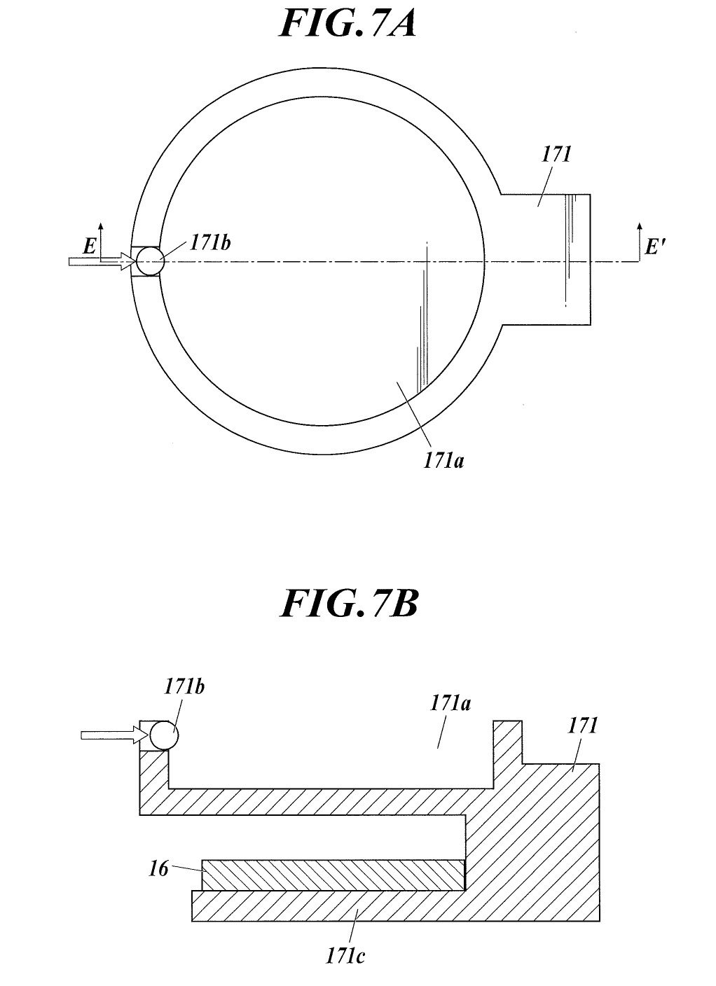

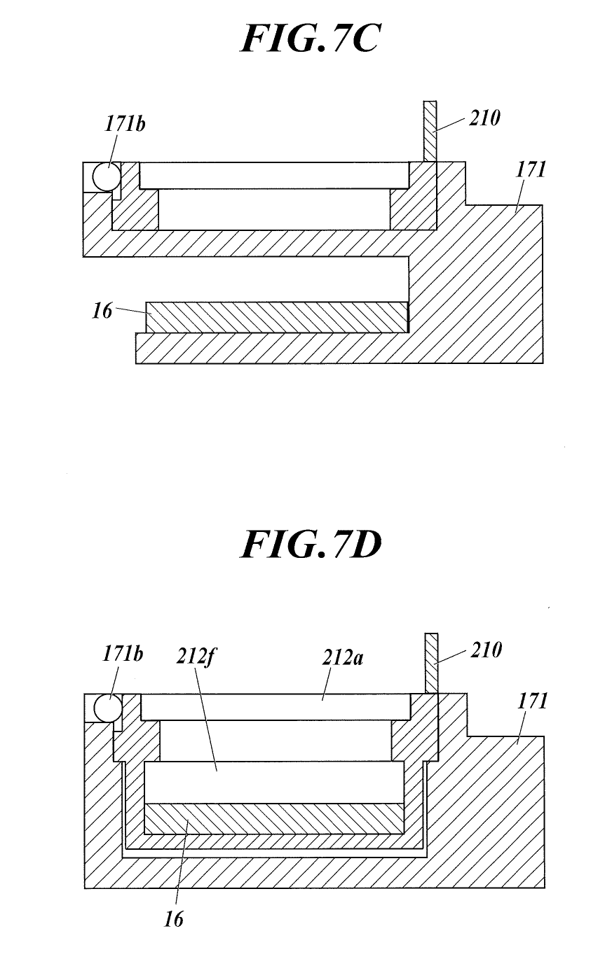

FIG. 7A is a planar view showing an enlarged holding portion 171 of the grating rotating section 210 in the holding section 17, and FIG. 7B is a cross sectional view along line E-E′ shown in FIG. 7A. FIG. 7C is a diagram showing a state of the holding section 17 holding the grating rotating section 210.

As shown in FIG. 7A and FIG. 7B, the holding portion 171 is provided with an opening section 171a which is a size to accurately fit with the rotating tray 212 of the grating rotating section 210 and which rotatably holds the rotating tray 212 and a tray fixing member 171b which fixes the rotating angle of the rotating tray 212. It is preferable that the space between the bottom section of the opening section 171a and the placing section of the X-ray detector 16 is hollow or includes a material with a high X-ray passing rate such as aluminum, carbon, etc., so that the passing of the X-ray is not interfered. The tray fixing member 171b includes a ball which is engaged to the opposing depressed section when any of the depressed section 212b to 212e is positioned to be opposite to the tray fixing member 171b, and a sliding guide (guide of the pressuring spring) which is not shown to guide the ball to the direction of the arrow shown in FIG. 7A and FIG. 7B. When the rotation of the rotating tray 212 is stopped with any of the depressed sections 212b to 212e in a position opposite to the tray fixing member 171b, the ball engages to the opposing depressed section by the sliding guide of the tray fixing member 171b, and the engaging of the ball is detected by the angle detecting sensor (any of SE1 to SE4) provided in the depressed section to output a detecting signal to the control section 181. With this, the control section 181 is able to detect the rotating angle of the rotating tray 212, in other words, the rotating angle of the first grating 14 and the second grating 15.

As shown in FIG. 7D, it is possible to provide an attaching section 212f of the X-ray detector 16 at the bottom portion of the opening section 212a of the rotating tray 212 so as to be able to rotate the first grating 14, the second grating 15, and the X-ray detector 16 as one. With this, there is no influence of the anisotropic aspect of the sharpness in the vertical and horizontal direction of the X-ray detector 16, and the sharpness in the horizontal and vertical direction of the reconstructed image can be basically stable regardless of the rotating angle of the first grating 14 and the second grating 15.

Returning to FIG. 1, in the X-ray detector 16, conversion elements are provided two-dimensionally to generate an electric signal according to the emitted X-ray, and the electric signals generated by the conversion elements are read as image signals. The pixel size of the X-ray detector 16 is 10 to 300 (μm), and preferably 50 to 200 (μm).

It is preferable that the position of the X-ray detector 16 is fixed to the holding section 17 so as to be in contact with the second grating 15. This is because as the distance between the second grating 15 and the X-ray detector 16 becomes larger, the moire image obtained by the X-ray detector 16 becomes blurred.

As the X-ray detector 16, it is possible to use an FPD (Flat Panel Detector). As the FPD, either of the following can be employed, an indirect conversion type which converts the X-ray to an electric signal with the photoelectric conversion through a scintillator or a direct conversion type which directly converts the X-ray to an electric signal.

In the indirect conversion type, each pixel is configured so that a photoelectric conversion element is provided two-dimensionally with a TFT (thin film transistor) under a scintillator plate of Csl, Gd₂O₂, etc. When the X-ray which enters the X-ray detector 16 is absorbed by the scintillator plate, the scintillator plate emits light. With the emitted light, charge is accumulated in each photoelectric conversion element, and the accumulated charge is read as the image signal.

In the direct conversion type, thermal deposition of amorphous selenium is performed to form an amorphous selenium film with a film thickness of 100 to 1000 (μm) on glass, and the amorphous selenium film and electrodes are vapor deposited on the array of the TFT provided two-dimensionally. When the amorphous selenium film absorbs the X-ray, voltage is liberated in the material as an electron hole pair, and the voltage signal between the electrodes is read by the TFT.

A capturing unit such as a CCD (Charge Coupled Device), an X-ray camera, etc. can be used as the X-ray detector 16.

A string of processing by the FPD in X-ray capturing is described.

First, the FPD resets the processing and removes unnecessary charge remaining after the previous capturing (reading). Then, the charge is accumulated at the timing of starting emission of the X-ray, and the charge accumulated at the timing when the emission of the X-ray ends is read as the image signal. Dark reading for offset correction is performed immediately after reset and after reading the image signal.

As shown in FIG. 8, the main section 18 includes a control section 181, an operation section 182, a display section 183, a communication section 184, and a storage section 185.

The control section 181 includes a CPU (Central Processing Unit), a RAM (Random Access Memory), etc. The control section 181 controls each section of the X-ray capturing apparatus 1 and performs various processing in coordination with programs stored in the storage section 185. For example, the control section 181 performs various processing such as a later described capturing control processing.

The operation section 182 includes an emitting switch, a key group used for input operation such as capturing conditions, and further includes a touch panel configured as one with the display of the display section 183. The operation section 182 generates an operation signal according to the above operation to output the signal to the control section 181.

The display section 183 displays an operation screen, behavior status of the X-ray capturing apparatus, or the like on the display according to the display control by the control section 181.

The communication section 184 includes a communication interface and communicates with the controller 5 on the network. For example, the communication section 184 transmits to the controller 5 the moire image read by the X-ray detector 16 and stored in the storage section 185.

The storage section 185 stores programs performed by the control section 181 and data necessary to perform the programs. Moreover, the storage section 185 stores the moire image obtained by the X-ray detector 16.

The controller 5 controls the capturing operation of the X-ray capturing apparatus 1 according to the operation by the operator. The controller 5 functions as the image processing section which creates a reconstructed image for diagnosis using the moire image obtained by the X-ray capturing apparatus 1.

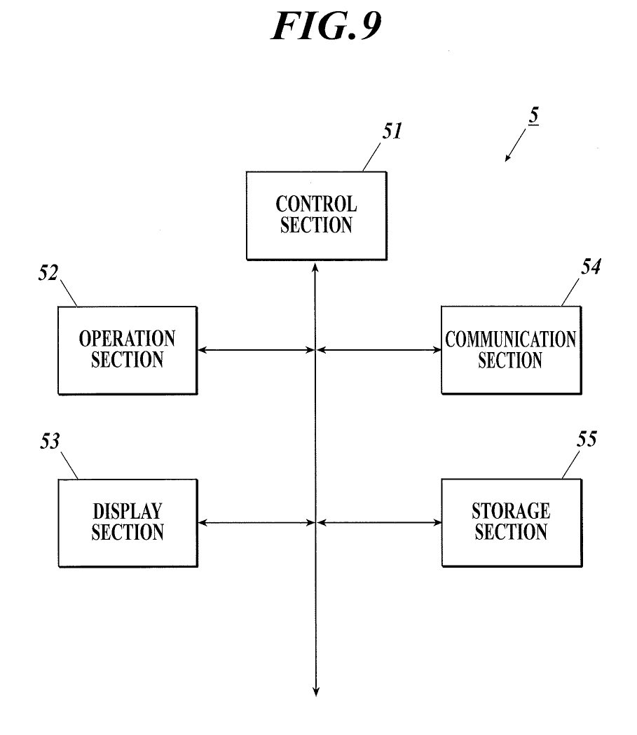

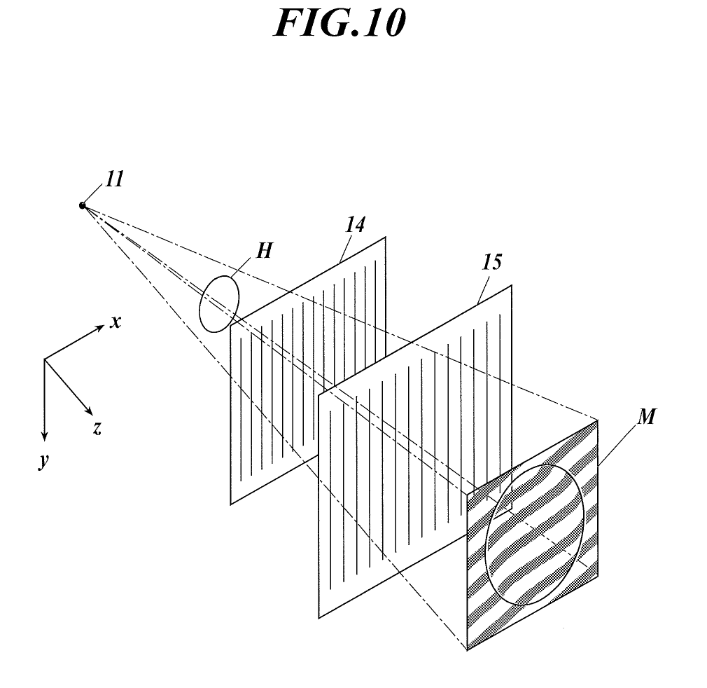

As shown in FIG. 9, the controller 5 includes a control section 51, an operation section 52, a display section 53, a communication section 54, and a storage section 55.

The control section 51 includes a CPU (Central Processing Unit), a RAM (Random Access Memory), etc. In coordination with the program stored in the storage section 55, the control section 51 performs various processing such as later described reconstructed image creating/displaying processing performed by a fringe scanning method and later described reconstructed image creating/displaying processing performed by a Fourier transformation method. By performing the reconstructed image creating/displaying processing by a fringe scanning method and the reconstructed image creating/displaying processing performed by a Fourier transformation method, the control section 51 creates at least two among an X-ray absorption image, a differential phase image, and a small angle scattering image based on the moire image obtained by capturing in the first capturing mode or the second capturing mode with the X-ray capturing apparatus and controls display of the created image on the display section 53. In other words, the control section 51 functions as an image processing section and a control section.

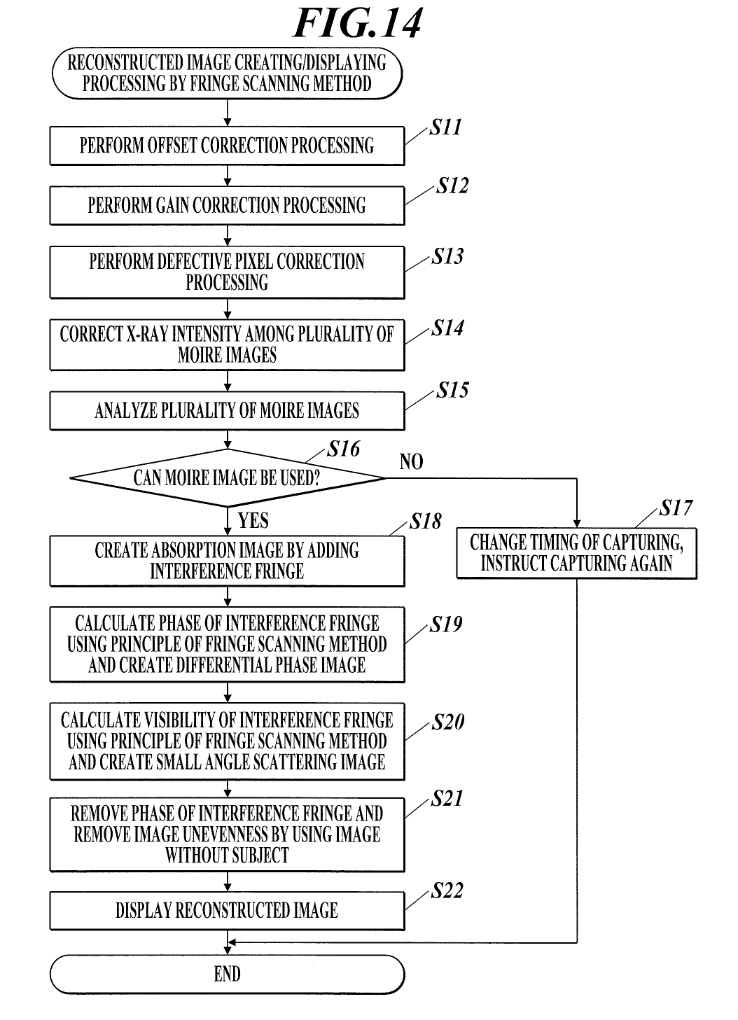

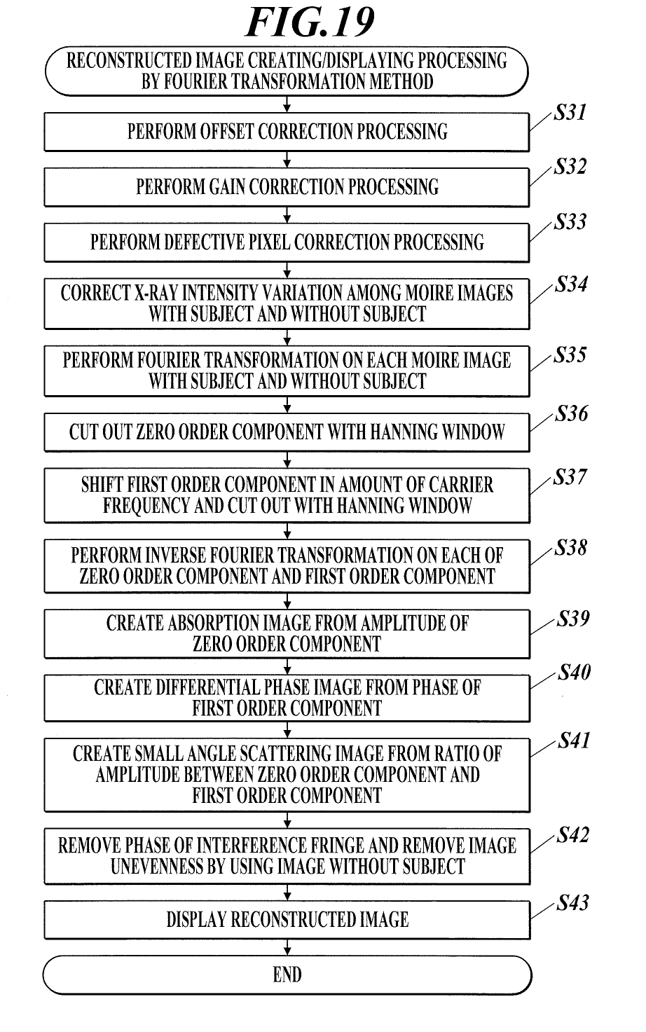

The operation section 52 includes a keyboard including cursor keys, numeral input keys and various function keys and a pointing device such as a mouse, etc. The operation section 52 outputs pressed signals of a key operated on the keyboard and operation signals of the mouse to the control section 51 as an input signal. A touch panel configured as one with the display of the display section 53 may be provided, and the operation signal according to the operation of the touch panel may be generated to be output to the control section 51. For example, according to the present embodiment, the operation section 52 is able to set the type of image displayed, timing of switching display of the image, etc. in step S22 of FIG. 14 or step S43 of FIG. 19, for each site or each user (physician).

For example, the display section 53 includes a monitor such as a CRT (Cathode Ray Tube), an LCD (Liquid Crystal Display), etc., and displays the operation screen, the behavior status of the X-ray capturing apparatus 1, the created reconstructed image, and the like according to the display control of the control section 51.

The communication section 54 includes a communication interface and communicates wired or wireless with the X-ray capturing apparatus 1 or the X-ray detector 16 on the network. For example, the communication section 54 transmits capturing conditions and control signals to the X-ray capturing apparatus 1, and receives moire images from the X-ray capturing apparatus 1 or the X-ray detector 16.

The storage section 55 stores programs performed by the control section 51 and data necessary to perform the programs. For example, the storage section 55 stores capturing order information showing a reserved order from RIS, HIS, etc. or a reservation device not shown. The capturing order information is information such as patient name, capturing site, capturing mode, etc.

Moreover, for example, the storage section 55 stores setting information set by the operation section 52, for example, type of image displayed in step S22 of FIG. 14 or step S43 of FIG. 19, switching timing of display of the image and the like, corresponded with site information and user ID.

The storage section 55 stores the moire image obtained by the X-ray detector 16 and the reconstructed image for diagnosis created based on the moire image corresponded with the capturing order information.

The storage section 55 stores a reference image (described in detail later) showing a typical example of a lesion corresponded with a lesion name, a type of image (fringe scanning method or Fourier transformation method, absorption image or differential phase image or small angle scattering image), and the like.

The storage section 55 stores in advance gain correction data, defective pixel map, etc. corresponding to the X-ray detector 16.

On the controller 5, when the display of the list of capturing order information is instructed by operation of the operation section 52, the control section 51 reads out the capturing order information from the storage section 55 to be displayed on the display section 53. When the capturing order information is specified on the operation section 52, the setting information of the capturing condition (including the capturing mode) according to the specified capturing order information, instruction to warm-up the X-ray source 11 and the like are transmitted to the X-ray capturing apparatus 1 by the communication section 54. With this, the capturing mode is set on the X-ray capturing apparatus 1. In other words, the controller 5 functions as the setting section to set the capturing mode. When the X-ray detector 16 is a cable-less cassette type FPD device, the control section 51 starts the device to a state in which capturing is possible from a sleep state to prevent wasting of the internal battery.

In the X-ray capturing apparatus 1, when setting information of the capturing condition and the like is received from the controller 5 by the communication section 184, the preparation for X-ray capturing is performed.

The X-ray capturing method (capturing method of the first capturing mode) by the Talbot-Lau interferometer of the X-ray capturing apparatus 1 is described.

As shown in FIG. 10, when the X-ray emitted from the X-ray source 11 passes the first grating 14, the X-ray which passes forms an image at a certain interval in the z-direction. This image is called a self image, and the effect of such self image forming is called the Talbot effect. The second grating 15 is positioned parallel to the position where the self image is formed, and the grating direction of the second grating 15 is slightly tilted from the position parallel to the grating direction of the first grating 14. Therefore, a moire image M can be obtained from the X-ray which passes through the second grating 15. When there is a subject H between the X-ray source 11 and the first grating 14, the phase of the X-ray is misaligned by the subject H, and the interference fringe on the moire image M is disordered at the edge of the subject H as the border, as shown in FIG. 10. The disorder of the interference fringe can be detected by processing the moire image M and the subject image can be imaged. This is the principle of the Talbot interferometer and the Talbot-Lau interferometer.

In the X-ray capturing apparatus 1, the multi-slit 12 is provided in a position near the X-ray source 11 between the X-ray source 11 and the first grating 14, and X-ray capturing by the Talbot-Lau interferometer is performed. It is presumed that when the Talbot-Lau interferometer is used, the X-ray source 11 is a desirable point source. However, in the actual capturing, since a focus with a focus diameter having a large size to a certain degree is used, the multi-slit 12 causes the X-ray to be emitted as if a plurality of light sources are aligned, and it is as if a plurality of light sources are generated. This is the X-ray capturing method by the Talbot-Lau interferometer, and even if the focus diameter is large to a certain degree, a Talbot effect similar to the Talbot interferometer can be achieved.

In the conventional Talbot-Lau interferometer, as described above, the multi-slit 12 is used for the purpose of increasing the light source and increasing the emission amount of radiation, and the first grating 14 or the second grating 15 were moved relatively in order to obtain a plurality of moire images. However, according to the present embodiment, instead of relatively moving the first grating 14 or the second grating 15, the positions of the first grating 14 and the second grating 15 are fixed and the multi-slit 12 is moved in relation with the first grating 14 and the second grating 15 to obtain a plurality of moire images at a certain cyclic interval.

When the moire image is obtained by the second capturing mode, the multi-slit 12 is not moved and capturing is performed once or the subject and the slit direction are rotated 90 degrees and capturing is performed twice.

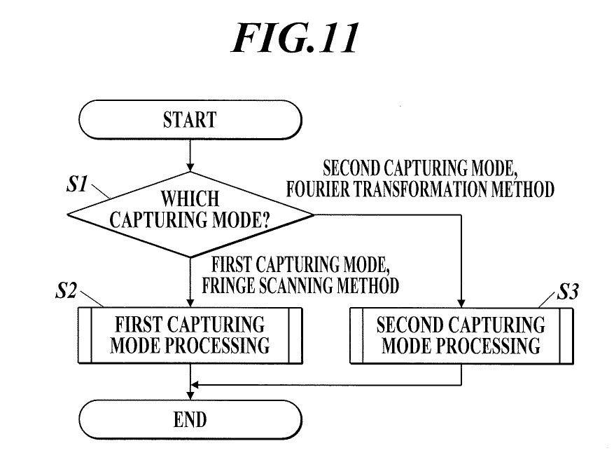

FIG. 11 is a flowchart showing a capturing control processing performed by the control section 181 of the X-ray capturing apparatus 1. The capturing control processing is performed by the control section 181 in coordination with the program stored in the storage section 185.

First, it is judged which capturing mode between the first capturing mode (fringe scanning method) and the second capturing mode (Fourier transformation method) is set based on the setting information received from the controller 5 (step S1). When it is judged that the first capturing mode is set (step S1; first capturing mode), the first capturing mode processing is performed (step S2). Alternatively, when it is judged that the second capturing mode is set (step S1; second capturing mode), the second capturing mode processing is performed (step S3).

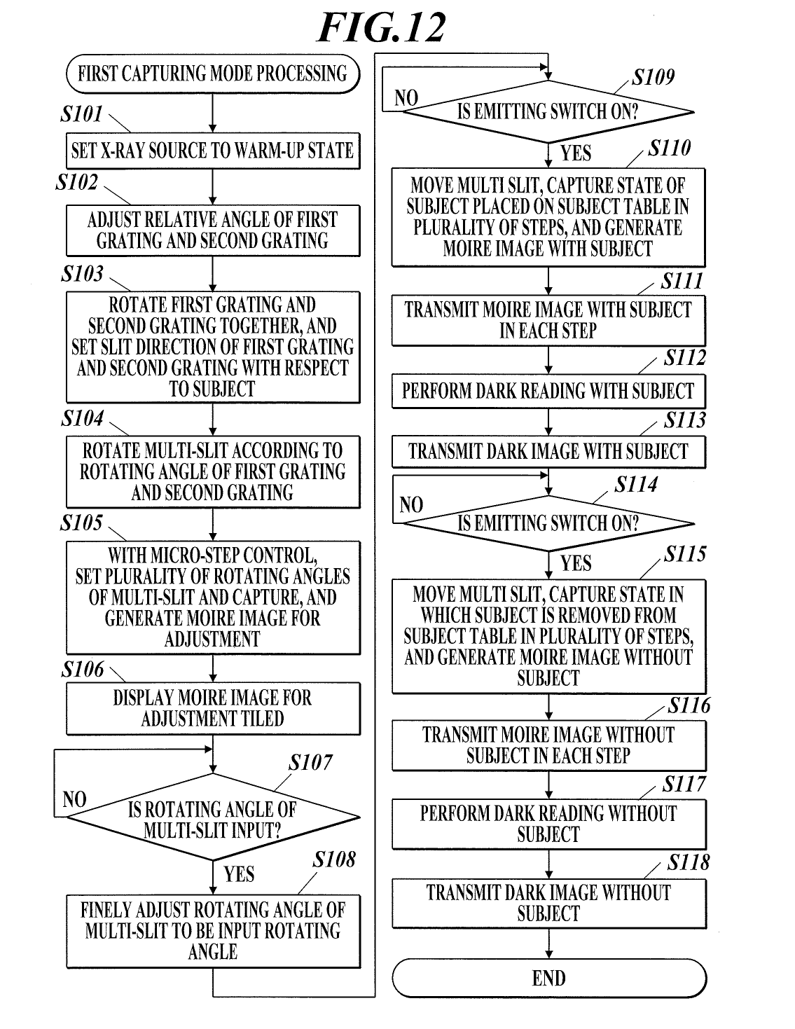

FIG. 12 is a flowchart showing the first capturing mode processing performed by the control section 181 of the X-ray capturing apparatus 1 in step S2 of FIG. 11. The first capturing mode processing is performed by the control section 181 in coordination with the program stored in the storage section 185.

Here, in the X-ray capturing of the first capturing mode, the above-described X-ray capturing method by the Talbot-Lau interferometer is used and in the reconstructing of the subject image, the fringe scanning method is used. In the X-ray capturing apparatus 1, the driving section 122 is controlled by the control section 181 to be driven and stopped to move the multi-slit 12 a plurality of steps where the steps are provided at an equal interval. Capturing is performed at each step to obtain the moire image of each step.

The number of steps is 2 to 20, and more preferably, 3 to 10. From the view point of obtaining a reconstructed image with high visibility in a short amount of time, five steps is preferable (Reference Document (1) K. Hibino, B. F. Oreb and D. I. Farrant, Phase Shifting for Non-Sinusoidal Wave Forms with Phase-Shift Errors, J. Opt. Soc. Am. A, Vol. 12, 761 to 768 (1995), Reference Document (2) A. Momose, W. Yashiro, Y. Takeda, Y. Suzuki and T. Hattori, Phase Tomography by X-ray Talbot Interferometery for Biological Imaging, Jpn. J. Appl. Phys., Vol. 45, 5254-5262 (2006)).

As shown in FIG. 12, first, the control section 181 switches the X-ray source 11 to a warm-up state (step S101).

Next, the relative angle adjusting section 213 of the grating rotating section 210 is controlled to rotate the first grating 14 so that the relative angle between the first grating 14 and the second grating 15 becomes suitable for the first capturing mode (the protrusion section 142a comes to a position in contact with the stopper 214). With this, the relative angle between the first grating 14 and the second grating 15 is adjusted (step S102).

Next, the first grating 14 and the second grating 15 are rotated as one by the operation of the operator, and the slit directions of the first grating 14 and the second grating 15 are set in relation with the subject (step S103). In other words, the operator such as the capturing technician, etc., rotates the handle 211 of the grating rotating section 210 to set the slit directions of the first grating 14 and the second grating 15 according to the positioning direction of the target configuration of the subject placed on the subject table 13. When the rotation of the handle 211 is stopped and the position is fixed by engaging to the spring-biased ball of the tray fixing member 171b, the detection signal is output from any one of the angle detecting sensors SE1 to SE4 to the control section 181, and the control section 181 obtains the rotating angle from the home position of the rotating tray 212 (in other words, the first grating 14 and the second grating 15) of the grating rotating section 210 corresponding to the set slit direction.

Next, according to the rotating angles of the first grating 14 and the second grating 15, the motor section 121a of the multi-slit rotating section 121 is controlled by a pulse to rotate the multi-slit 12 according to the rotating angles of the first grating 14 and the second grating 15 (step S104). For example, the pulse motor of the motor section 121a is controlled to rotate at once so that the rotating angle from the home position of the multi-slit 12 becomes near the rotating angle of the rotating tray 212 (for example, when the rotating tray 212 is set to 30°, about 29°).

Next, the motor section 121a is switched to micro-step precise control, the multi-slit 12 is rotated little by little and capturing is performed at a plurality of rotating angles to generate a plurality of moire images for adjustment (step S105). For example, when the rotating tray 212 is set to 30°, the multi-slit 12 is set to three rotating angles of 29.5°, 30°, and 30.5° and the X-ray with low amount of radiation is emitted by the X-ray source 11 to perform capturing. With this, three moire images for adjustment are obtained. In step S105, capturing is performed in a state without placing the subject on the subject table 13.

The plurality of moire images for adjustment which are captured are corresponded with the rotating angle of the multi-slit 12 and displayed tiled on the display section 183 (step S106).

Here, as described above, the relative angle between the first grating 14 and the second grating 15 is adjusted in step S102 so that the number of interference fringes becomes smallest. Therefore, in step S103, the first grating 14 and the second grating 15 are rotated by the rotation of the rotating tray 212 maintaining the relative angle. However, when the rotating tray 212 in which the first grating 14 and the second grating 15 are placed rotates and the relative angle made by the multi-slit 12 with the first grating 14 and the second grating 15 changes, the clarity of the interference fringe (in other words, moire) changes. Therefore, it is necessary to adjust the relative angle made by the multi-slit 12 with the first grating 14 and the second grating 15, in other words, the rotating tray 212 in which the first grating 14 and the second grating 15 are placed.

Typically, as the relative angle between the multi-slit 12 and the first grating 14 becomes smaller, a moire image with the interference fringe with higher clarity can be obtained. However, since the multi-slit 12 is positioned near the X-ray source 11 which is a heat generating section, the multi-slit 12 easily receives influence of the heat. Therefore, considering the deformation, etc. of the multi-slit 12, in addition to rotating the multi-slit 12 in the same degree as the rotating tray 212, it is effective to perform fine adjustment in steps S105 to S108 by micro-step driving of the motor section 121a.

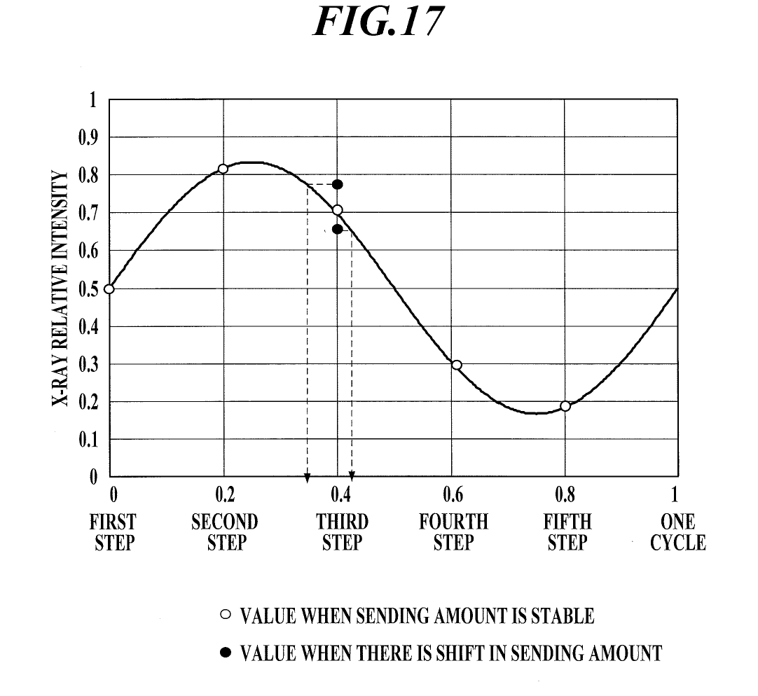

The operator observes the moire image displayed on the display section 183 in step S106, and the rotating angle with the clearest interference fringe is selected as the rotating angle used in capturing. Here, the clarity of the interference fringe is observed by sight of the operator. However, the clarity degree showing the degree of clarity of the interference fringe can be represented by the formula below, where a local maximum value is to be MAX and a local minimum value is to be MIN in a later described sine curve (see FIG. 17). Instead of by the operator, the rotating degree so that the clarity degree of the interference fringe is the highest value can be set automatically by a program using the clarity degree of the interference fringe.

clarity degree of interference fringe=(MAX−MIN)/(MAX+MIN)=amplitude/average

When the rotating angle of the multi-slit 12 is input on the operation section 182 (step S107; YES), the motor section 121a is driven again, and the position of the multi-slit 12 is finely adjusted so that the rotating angle of the multi-slit 12 from the home position becomes the input rotating angle (step S108).

After adjusting the rotating angle of the multi-slit 12, the subject is placed on the subject table 13, and the emitting switch is turned ON by the operator (step S109; YES). Then, the multi-slit 12 is moved in the slit array direction by the driving section 122, capturing of a plurality of steps is performed, and the plurality of moire images with the subject is generated (step S110).

First, the X-ray source 11 starts emitting the X-ray in a state where the multi-slit 12 is stopped. In the X-ray detector 16, after reset, the charge is accumulated at the timing of the X-ray emission, and the accumulated charge is read as the image signal at the timing when the X-ray emission stops. This is capturing for one step. At the timing when capturing for one step ends, the driving section 122 is started by the control of the control section 181 and the movement of the multi-slit 12 starts. After the multi-slit 12 is moved a predetermined amount, the driving section 122 is stopped to stop the movement of the multi-slit 12, and capturing of the next step is performed. With this, the movement and stopping of the multi-slit 12 is repeated for the predetermined number of steps, and when the multi-slit 12 is stopped, the emission of the X-ray and reading of the image signal is performed. The read image signal is output to the main section 18 as the moire image.

For example, the slit cycle of the multi-slit 12 is set to 22.8 (μm) and the capturing of five steps is performed in 10 seconds. The capturing is performed each time the multi-slit 12 moves 4.56 (μm), which is ⅕ of the slit cycle, and stops.

If the second grating 15 (or the first grating 14) is moved as in conventional methods, the slit cycle of the second grating 15 becomes comparatively small, and the movement amount of each step also becomes small, whereas the slit cycle of the multi-slit 12 is comparatively larger than the second grating 15 and the movement amount of each step also becomes large. For example, the movement amount for each step for the second grating 15 with the slit cycle of 5.3 (μm) is 1.06 (μm), whereas the movement amount for the multi-slit 12 with the slit cycle of 22.8 (μm) is 4.56 (μm), and this is about four times larger. When the same driving transmission system (including driving source, decelerating transmission system) is used, and the driving section 122 is started and stopped repeatedly to perform capturing for capturing of each step, the ratio of the movement amount error due to backlash, etc. of the driving section 122 in starting and stopping which consists the actual amount of movement corresponding to the control amount (driving pulse number) of the pulse motor (driving source) for movement becomes smaller in a method as described in the present embodiment where the multi-slit 12 is moved. This shows that, the moire image along the later described sine curve is easy to obtain, and a high-definition reconstructed image can be obtained even if the start and stop is repeated. Alternatively, if the image by the conventional method is suitable enough for diagnosis, this shows that it is possible to ease the accuracy (specifically, starting character and stopping character) of the entire driving transmission system including the motor (driving source). Consequently, it is possible to reduce costs of components of the driving transmission system.

When the capturing of each step finishes, the moire image of each step is transmitted from the communication section 184 of the main section 18 to the controller 5 (step S111). The moire image including the subject is transmitted one image at a time from the main section 18 to the controller 5 until the capturing in each step ends.

Next, the dark reading in the X-ray detector 16 is performed, and a dark image (offset correction data) for correction of the image data including the subject is obtained (step S112). The dark reading is performed at least once. Alternatively, the dark reading can be performed a plurality of times to obtain the average value as the dark image. The dark image is transmitted from the communication section 184 to the controller 5 (step S113). The offset correction data based on the dark reading is also used in the correction of each moire image signal.

Regarding obtaining the dark image, the step of dark reading can be performed to generate offset correction data exclusive for each step after the step of obtaining the moire image.

Next, the process waits for the operator to turn ON the emitting switch (step S114). Here, the operator removes the subject from the subject table 13 and evacuates the patient to create a moire image without the subject. When the preparation for capturing without the subject is complete, the emitting switch is pressed.

When the emitting switch is pressed (step S114; YES), the driving section 122 drives the multi-slit 12 to move in the slit array direction, a plurality of steps of capturing without the subject is performed, and a plurality of moire images without the subject is generated (step S115). When the capturing of each step is finished, the communication section 184 of the main section 18 transmits the moire image of each step to the controller 5 (step S116). The moire image without the subject is transmitted one image at a time from the main section 18 to the controller 5 by the communication section 184 each time the capturing of each step ends.

Next, the dark reading is preformed in the X-ray detector 16, and the dark image without the subject is obtained (step S117). The dark reading is performed at least once. Alternatively, the dark reading can be performed a plurality of times, and the average value can be obtained as the dark image. The dark image is transmitted from the communication section 184 to the controller 5 (step S118), and the string of capturing for one capturing order ends.

Regarding obtaining the dark image, the step of dark reading can be performed to generate offset correction data exclusive for each step after obtaining the moire image in each step.

For accuracy, it is most preferable to perform the capturing of the plurality of moire images without the subject and the dark reading immediately after the capturing with the subject. However, in order to reduce time of reconstructing the subject image, it is possible to use data obtained in advance such as before starting.

In the controller 5, when the moire image is received by the communication section 54, the received moire image is stored in the storage section 55 corresponded with the capturing order information specified when the capturing starts.

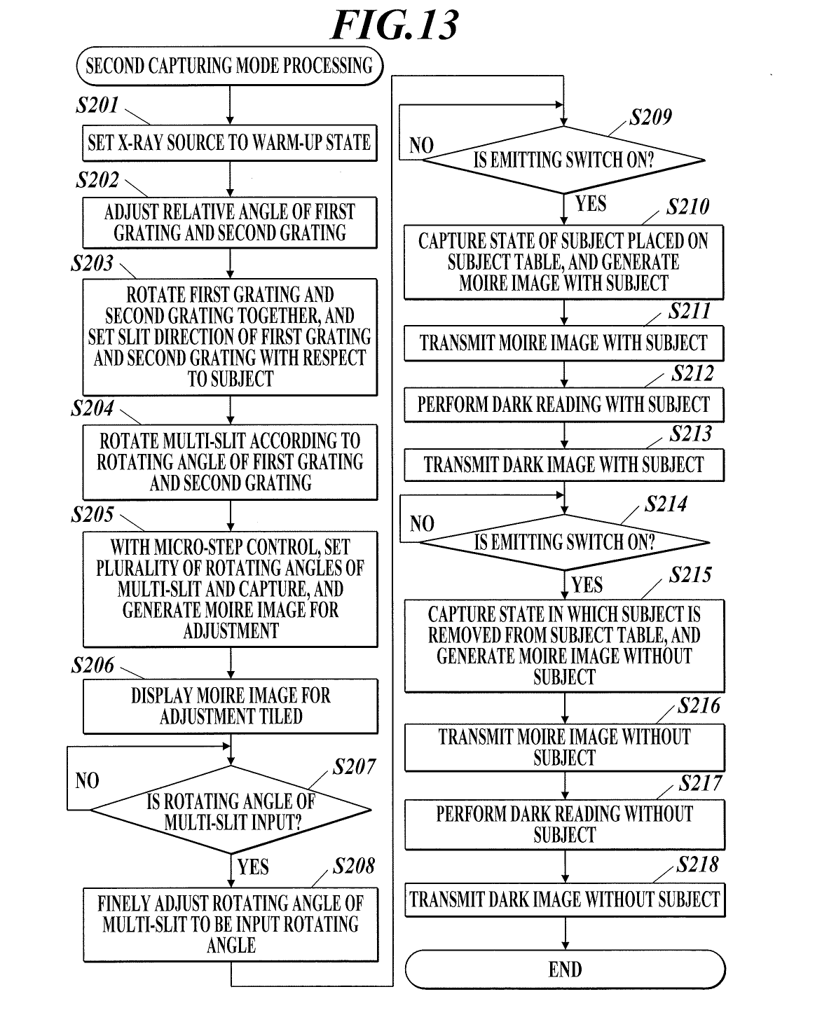

FIG. 13 is a flowchart showing a second capturing mode performed by the control section 181 of the X-ray capturing apparatus 1 in step S3 of FIG. 11. The second capturing mode processing is performed by the control section 181 in coordination with the program stored in the storage section 185.

As shown in FIG. 13, first, the control section 181 switches the X-ray source 11 to the warm-up state (step S201).

Next, the relative angle adjusting section 213 of the grating rotating section 210 is controlled and the relative angle between the first grating 14 and the second grating 15 is adjusted so as to be suitable for the second capturing mode (so that the protrusion section 142a is brought to a position rotated a predetermined angle from the home position) (step S202).

Next, the processing of step S203 to step S208 is performed. The processing of steps S203 to S208 is similar to the processing described in steps S103 to S108 of FIG. 12, therefore the description is incorporated herein.

When the subject is placed on the subject table 13 and the emitting switch is turned ON by the operator (step S209; YES), the capturing is performed to generate a moire image including the subject (step S210). In other words, the radiation is emitted from the X-ray source 11, and reading is performed in the X-ray detector 16. In the second capturing mode, the driving section 122 remains stopped, and the capturing of one image is performed without moving the multi-slit 12.

When the capturing ends, the moire image obtained by capturing is transmitted from the communication section 184 of the main section 18 to the controller 5 (step S211).

Next, the dark reading is performed in the X-ray detector 16, and the dark image (offset correction data) for correction of image data with the subject is obtained (step S212).

Next, the dark reading in the X-ray detector 16 is performed, and a dark image (offset correction data) for correction of the image data including the subject is obtained (step S212). The dark reading is performed at least once. Alternatively, the dark reading can be performed a plurality of times to obtain the average value as the dark image. The dark image is transmitted from the communication section 184 to the controller 5 (step S213). The offset correction data based on the dark reading is also used in the correction of each moire image signal.

Next, the process waits for the operator to turn ON the emitting switch (step S214). Here, the operator removes the subject from the subject table 13 and evacuates the patient to create the moire image without the subject. When the preparation for capturing without the subject is complete, the emitting switch is pressed.

When the emitting switch is pressed (step S214; YES), capturing without the subject is performed, and the moire image without the subject is generated (step S215). Similar to step S210, in step S215, the driving section 122 remains stopped and capturing of one image is preformed without moving the multi-slit 12.

When the capturing ends, the moire image is transmitted from the communication section 184 of the main section 18 to the controller 5 (step S216).

Next, the dark reading is preformed in the X-ray detector 16, and the dark image without the subject is obtained (step S217). The dark reading is performed at least once. Alternatively, the dark reading can be performed a plurality of times, and the average value can be obtained as the dark image. The dark image is transmitted from the communication section 184 to the controller 5 (step S218), and the string of capturing for one capturing order ends.

For accuracy, it is most preferable to perform the capturing of the plurality of moire images without the subject and the dark reading immediately after the capturing with the subject. However, in order to reduce time of reconstructing the subject image, it is possible to use data obtained in advance such as before starting.

In the control section 51 of the controller 5, when the moire image is received by the communication section 54, if the capturing mode set in the capturing order information of the present processing target is the first capturing mode, the reconstructed image creating/displaying processing by the fringe scanning method is performed, and if the capturing mode is the second capturing mode, the reconstructed image creating/displaying processing by the Fourier transformation method is performed.

FIG. 14 is a flowchart showing the reconstructed image creating/displaying processing by the fringe scanning method performed by the control section 51. The reconstructed image creating/displaying processing by the fringe scanning method is performed by the control section 51 in coordination with the program stored in the storage section 55.

First, in steps S11 to S13, correction processing to correct the variation in each pixel of the X-ray detector 16 is performed for the plurality of moire images with the subject and the plurality of moire images without the subject. Specifically, offset correction processing (step S11), gain correction processing (step S12), and defective pixel correction processing (step S13) are performed.

In step S11, the offset correction is performed on each moire image with the subject based on the dark image for correction of image data with the subject. The offset processing is performed on each moire image without the subject based on the dark image for correction of image data without the subject. In step S12, the gain correction data corresponding to the X-ray detector 16 used in capturing is read from the storage section 55, and based on the read gain correction data, the gain correction is performed on each moire image.

In step S13, the defective pixel map (data showing defective pixel position) corresponding to the X-ray detector 16 used in the capturing is read from the storage section 55, and the pixel value (signal value) of the position shown in the defective pixel position map of each moire image is calculated by interpolation with the surrounding pixels.

Next, the X-ray intensity variation correction (trend correction) is performed among the plurality of moire images (step S14). In the fringe scanning method, one reconstructed image is created based on the plurality of moire images. Therefore, when there is fluctuation (variation) in the X-ray intensity emitted in the capturing of each moire image, a sophisticated reconstructed image cannot be obtained, and there is a possibility that a slight change in the signal is overlooked. Therefore, in step S14, processing is performed to correct the difference in signal value due to the variation of the X-ray intensity in capturing the plurality of moire images.

The specific processing may be any method described below, such as a method of correction using the signal value of one predetermined pixel in each moire image, a method of correction of the signal value difference in the predetermined direction of the X-ray detector 16 among moire images (one-dimensional correction), and a method of correction of the signal value difference in the two-dimensional direction among moire images (two-dimensional correction).

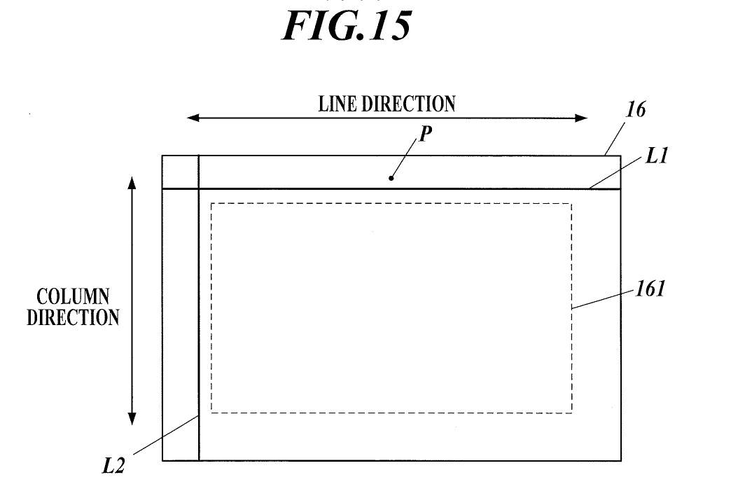

As the method of correction using the signal value of one pixel, first, the method obtains the signal value of the pixel in the predetermined position P corresponding to the direct X-ray region outside the moire image region (subject position region) 161 of the X-ray detector 16 for each of the plurality of moire images shown in FIG. 15. Next, the first moire image (for example, the moire image first captured with the subject) is standardized with the average signal value of the pixel in the obtained position P among the second image and after, and the correction coefficient of each of the second moire image and after is calculated based on the value of the position P after standardizing. Then, the correction coefficient is multiplied to each of the second moire image and after, and the X-ray intensity variation is corrected. With this correction method, it is possible to easily correct the entire variation of the X-ray intensity among capturing. It is possible to provide a detecting unit such as a sensor on a back side of the X-ray detector 16 to detect the X-ray emission amount and it is possible to correct the signal value difference among the moire images due to X-ray intensity variation in capturing based on the X-ray emission amount output from the detecting section when each moire image is captured.

In the one-dimensional correction, first, the average signal value of the pixels in a predetermined line L1 (a line is a reading line direction in the X-ray detector 16) is calculated for each of the plurality of moire images. Next, the first moire image is standardized with the average signal value of the pixels of the second moire image and after, and the correction coefficient in the line direction of each of the second moire image and after is calculated based on the signal value of each pixel of the standardized line L1 and the line L1 of the second moire image and after. Then, the correction coefficient according to the position in the line direction is multiplied to each of the second moire image and after to correct the X-ray intensity variation in the line direction. With this correction method, it is possible to easily correct the variation of the X-ray intensity in the one-dimensional direction among the capturing. For example, in one capturing, when the emission timing by the X-ray source 11 and the reading timing of the X-ray detector 16 does not match, it is possible to correct the X-ray intensity variation in the reading line direction of the X-ray detector 16 caused by the above.

In the two-dimensional correction, first, the average signal value of the pixels in predetermined line L1 and column L2 (a column is a direction orthogonal to the reading line direction in the X-ray detector 16) is calculated for each of the plurality of moire images. Next, the first moire image is standardized with the average signal value of the pixels of the line L1 of the second moire image and after, and the correction coefficient in the line direction of each of the second moire image and after is calculated based on the signal value of each pixel of the standardized line L1 and the line L1 of the second moire image and after. Similarly, the first moire image is standardized with the average signal value of the pixels of the column L2 of the second moire image and after, and the correction coefficient in the column direction of each of the second moire image and after is calculated based on the signal value of each pixel of standardized column L2 and column L2 of the second moire image and after. Then, the correction coefficient in the line direction and the column direction are multiplied to each other, and the correction coefficient of each pixel in each of the second moire image and after is calculated. Then, the correction coefficient in the line direction and the column direction is multiplied to each pixel to correct the X-ray intensity variation in the two-dimensional direction. With this correction method, it is possible to easily correct the variation of the X-ray intensity in the two-dimensional direction among the capturing.

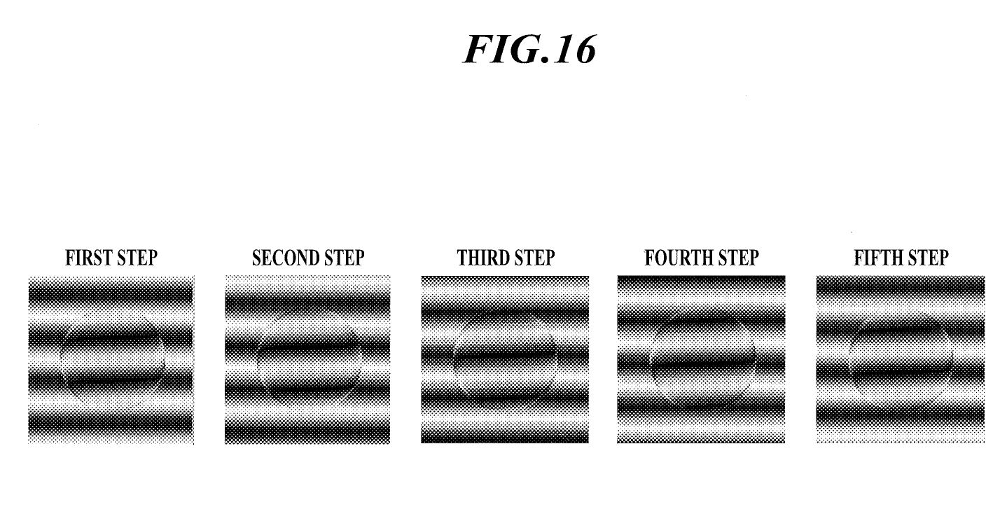

Next, the moire image is analyzed (step S15), and it is determined whether the moire image can be used in creating the reconstructed image (step S16). When it is possible to move the multi-slit 12 at a stable sending amount with an ideal sending accuracy, as shown in FIG. 16, five moire images for one slit cycle of the multi-slit 12 can be obtained in capturing of five steps. Since the moire image of each step is a result of fringe scanning at a certain cyclic interval which is 0.2 cycles, when any one pixel of each moire image is observed, the X-ray relative intensity obtained by normalizing the signal value draws a sine curve as shown in FIG. 17. Therefore, the controller 5 obtains the X-ray relative intensity by observing a certain pixel in the moire image of each step. If the X-ray relative intensity obtained from each moire image forms a sine curve as shown in FIG. 17, a moire image with a certain cyclic interval can be obtained, and it is possible to judge that the moire image can be used for creating the reconstructed image.

The form of the sine curve depends on opening width of the multi-slit 12, cycle of the first grating 14 and the second grating 15, and the distance between the first grating and the second grating. Moreover, with a coherent ray such as a radiation ray, the shape becomes a triangular wave shape, however, since the X-ray acts as a quasi-coherent ray due to the multi-slit effect, a sine curve is drawn. The analysis in step S15 is performed on each of the moire image with the subject and the moire image without the subject.

When there is a moire image where a sine curve cannot be formed among the moire images of each step, it is judged that the moire image cannot be used for creating the reconstructed image (step S16; NO), and control information to instruct changing the capturing timing and performing the capturing again is transmitted from the controller 5 to the X-ray capturing apparatus 1 (step S17). For example, as shown in FIG. 17, the proper cycle of the third step is 0.4 cycles, however, the cycle is off the proper cycle and the obtained moire image is 0.35 cycles, then it is assumed that the reason is decrease in sending accuracy of the driving section 122 (for example, overlapping noise to the driving pulse of the pulse motor, etc). Therefore, it is instructed to advance the capturing timing at 0.05 cycles and to capture only the third step again. Alternatively, it is possible to instruct to capture all five steps again, and the capturing time can be advanced at 0.05 cycles for only the third step. When the moire image of all five steps do not match the sine curve a predetermined amount each, it is possible to instruct increase or decrease of the driving pulse number from the start to stop of the driving section 122.

In the X-ray capturing apparatus 1, the timing of capturing is adjusted according to the control information and the capturing is performed again.

When it is judged that the moire image can be used in creating the reconstructed image (step S16; YES), the plurality of moire images with the subject and without the subject are each used to create a reconstructed image with the subject and the reconstructed image without the subject (step S18 to step S20).

Specifically, the absorption image (X-ray absorption image) is created by adding the interference fringe of the plurality of moire images (step S18). Moreover, the phase of the interference fringe is calculated using the principle of the fringe scanning method to create the differential phase image (step S19). The Visibility (Visibility=2×amplitude÷average value) of the interference fringe is calculated using the principle of the fringe scanning method to create the small angle scattering image (step S20).

Next, the reconstructed image without the subject is used to perform correction processing in order to remove the phase of the interference fringe to remove the image unevenness (artifact) from the reconstructed image with the subject (step S21). The processing of step S21 includes processing to remove image unevenness (artifact) such as the X-ray amount distribution unevenness due to change of the slit directions of the multi-slit 12, the first grating 14, and the second grating 15 in capturing, radiation amount distribution unevenness due to variation in manufacturing the slit, and unevenness of mainly the subject holder 130 being imaged in the image.

For example, when the reconstructed image with the subject is the differential phase image, processing to subtract the corresponding (same position pixel) signal value of the differential phase image without the subject from the signal value of each pixel of the differential phase image with the subject is performed. (See Publicly Known Document (A); Timm Weitkamp, Ana Diazand, Christian David, Franz Pfeiffer, and Marco Stampanoni, Peter Cloetens and Eric Ziegler, X-ray Phase Imaging with a Grating Interferometer, OPTICSEXPRESS, Vol. 13, No. 16, 6296-6004 (2005), Publicly Known Document (B); Atsushi Momose, Wataru Yashiro, Yoshihiro Takeda, Yoshio Suzuki, and Tadashi Hattori, Phase Tomography by X-ray Talbot Interferometery for Biological Imaging, Japanese Journal of Applied Physics, Vol. 45, No. 6A, 2006, pp. 5254-5262 (2006)).

When the reconstructed image with the subject is the absorption image or the small angle scattering image, as described in Publicly Known Document (C), processing to divide the signal value of each pixel of the reconstructed image with the subject with the signal value of the corresponding pixel of the reconstructed image without the subject is performed (Publicly Known Document (C); F. Pfeiffer, M. Bech, O. Bunk, P. Kraft, E. F. Eikenberry, C. H. Broennimann, C. Grunzweig, and C. David, Hard-X-ray Dark-Field Imaging Using a Grating Interferometer, Nature Materials Vol. 7, 134-137 (2008)).

In the above processing, it is preferable to be able to remove the influence of not only the X-ray amount distribution unevenness due to the change in slit directions of the gratings of the multi-slit 12, the first grating 14, and the second grating 15 and the characteristic of the subject table, but also the variation in characteristics of each pixel of the X-ray detector 16 used in capturing. Therefore, even if the slit direction can be changed according to the subject, the positioning direction of the X-ray detector 16 with respect to the subject can be fixed (position is not changed). The displayed direction of the subject in the reconstructed image displayed on the controller 5 is always the same direction on the controller display screen. Therefore, when interpretation to compare with the past image in followed observation, etc. is performed, there is no need to operate the controller 5 to align the direction of the reconstructed image, which is preferable.

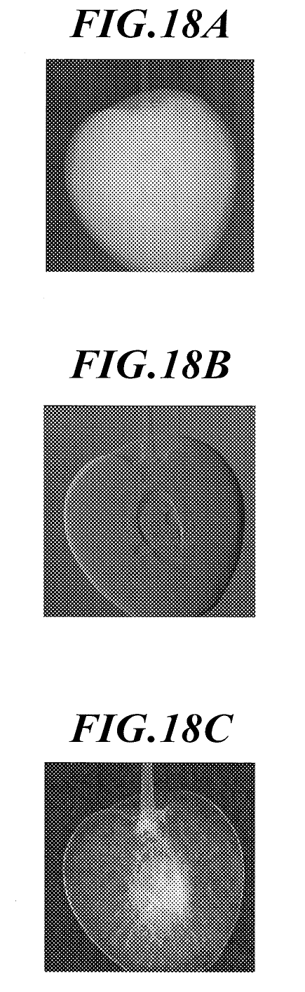

FIG. 18A to FIG. 18C shows an example of a reconstructed image created by the fringe scanning method based on the moire image captured with a cherry as the subject. FIG. 18A is the absorption image, FIG. 18B is the differential phase image, and FIG. 18C is the small angle scattering image.

As shown in FIG. 18A, the absorption image shows change in the large configuration of the subject. As shown in FIG. 18B, the differential phase image shows phase change of a tissue border of the subject. As shown in FIG. 18C, the small angle scattering image shows the scattering in the tissue of the subject.

When the processing of step S21 ends, the created reconstructed image is displayed on the display section 53 (step S22). The display mode of the reconstructed image in step S22 is described later.

Next, creating and displaying of the reconstructed image by the Fourier transformation method is described.

FIG. 19 is a flowchart showing reconstructed image creating/displaying processing by the Fourier transformation method performed by the control section 51. The reconstructed image creating/displaying processing by the Fourier transformation method is performed by the control section 51 in coordination with the program stored in the storage section 55.

First, in steps S31 to S33, correction processing to correct the variation in each pixel of the X-ray detector 16 is performed for the plurality of moire images with the subject and the plurality of moire images without the subject. Specifically, offset correction processing (step S31), gain correction processing (step S32), and defective pixel correction processing (step S33) are performed. The content of the processing is the same as those described in steps S11 to S13 of FIG. 14, therefore the description is incorporated herein.

Next, the X-ray intensity variation correction (trend correction) among the moire images with the subject and the moire images without the subject is performed (step S34). The specific content of processing of the X-ray intensity variation correction is similar to that described in step S14 of FIG. 14, therefore the description is incorporated herein.

Next, in the processing after step S35, the reconstructed image of the subject is created by the Fourier transformation method. The reconstructed image can be created by the Fourier transformation method using publically known methods (see Non-Patent Document 1).

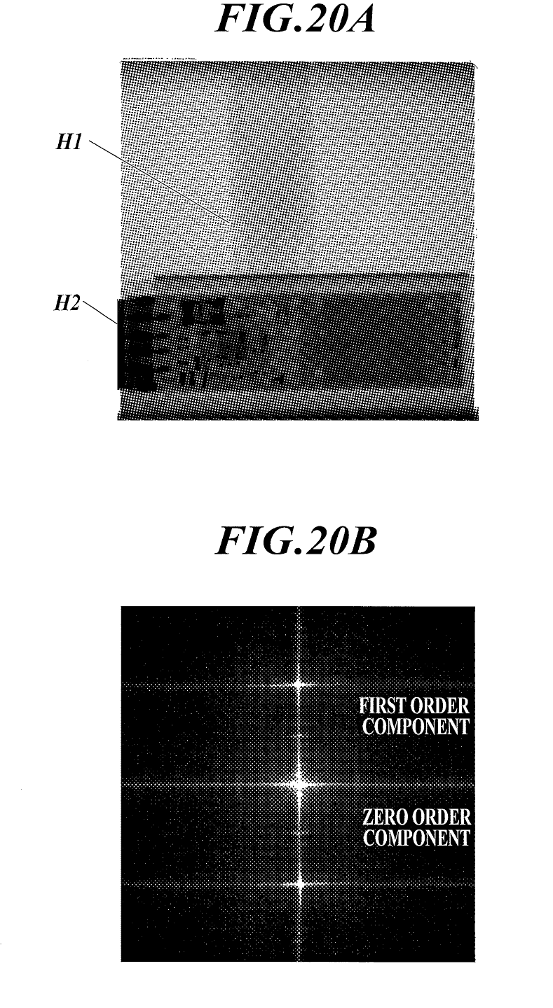

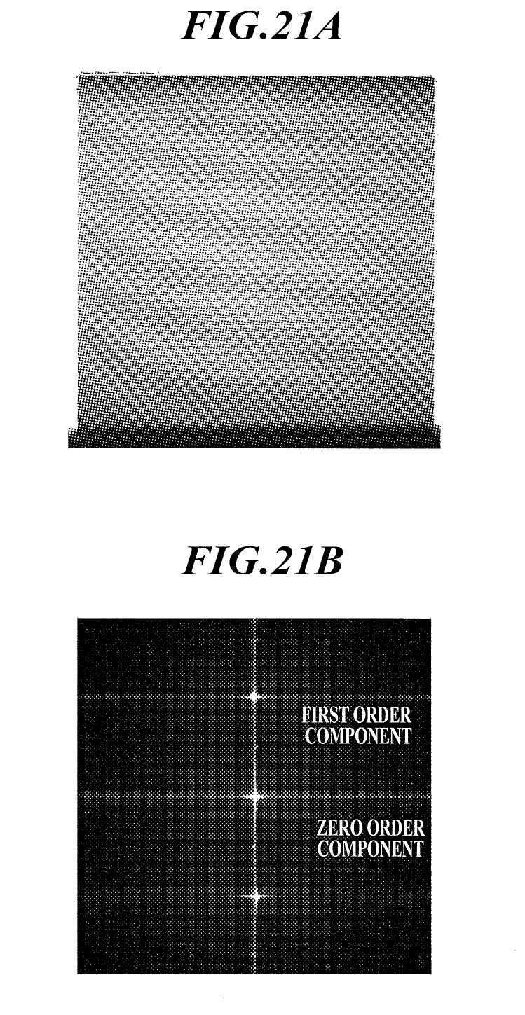

First, Fourier transformation (two-dimensional Fourier transformation) is performed on each of the corrected moire image with the subject and the corrected moire image without the subject (step S35). FIG. 20A shows an example of a moire image with the subject captured in the second capturing mode. In FIG. 20A, H1 is a marker, and H2 is a USB memory. FIG. 20B shows a result of two-dimensional Fourier transformation performed on the moire image of FIG. 20A. FIG. 21A shows an example of a moire image without the subject captured in the second capturing mode. FIG. 21B shows a result of two-dimensional Fourier transformation performed on the moire image of FIG. 21A. Since the calculated result after Fourier transformation is a complex number, FIG. 20B and FIG. 21B display a norm (amplitude) of a real part and an imaginary part.

As shown in FIG. 20B and FIG. 21B, when Fourier transformation is performed on one moire image, a low frequency component (called zero order component) and component near the frequency of the interference fringe (called first order component) or in addition to the zero order component and the first order component, a high frequency component (depending on the interference of the X-ray capturing apparatus 1) can be obtained aligned. The aligning direction of the zero order component and the first order component is related to the direction of the fringe of the moire image and is substantially at a right angle to the direction of the fringe of the moire image.

Described below is the relation of directions between the directions of the gratings of the multi-slit 12, the first grating 14, and the second grating 15 (slit direction), and the direction of the alignment of the interference fringe and the zero order component and the first order component.

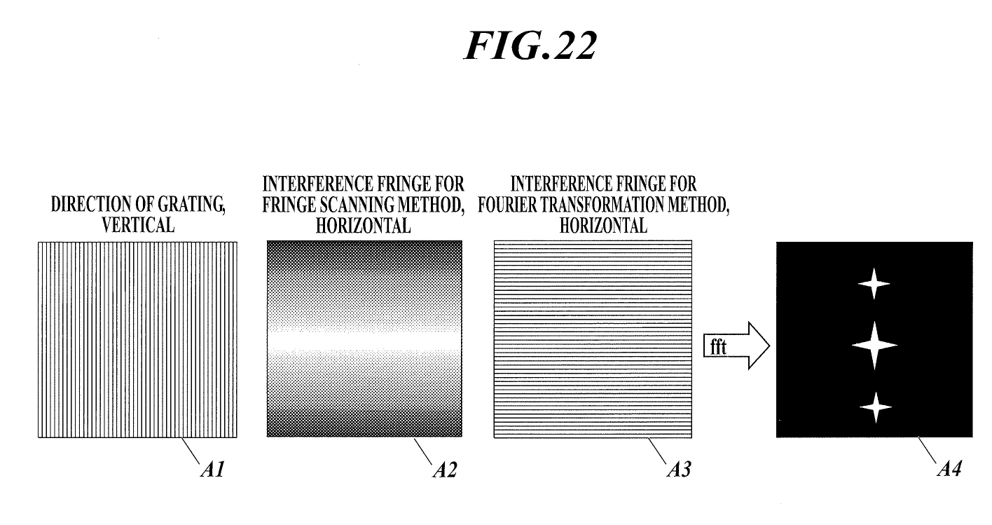

For example, as shown in A1 of FIG. 22, when the directions of the gratings of the multi-slit 12, the first grating 14, and the second grating 15 are vertical, the fringe of the moire image (an image which can be obtained by slightly tilting the second grating 15 with respect to the first grating 14) for the fringe scanning method becomes horizontal as shown in A2 of FIG. 22. As shown in A3 of FIG. 22, the fringe of the moire image for Fourier transformation (image obtained by further tilting the second grating) becomes a horizontal fringe finer compared to A2 of FIG. 22. As shown in A4 of FIG. 22, the image where the Fourier transformation is performed on the moire image for Fourier transformation becomes an image with the zero order component and the first order component aligned vertically.

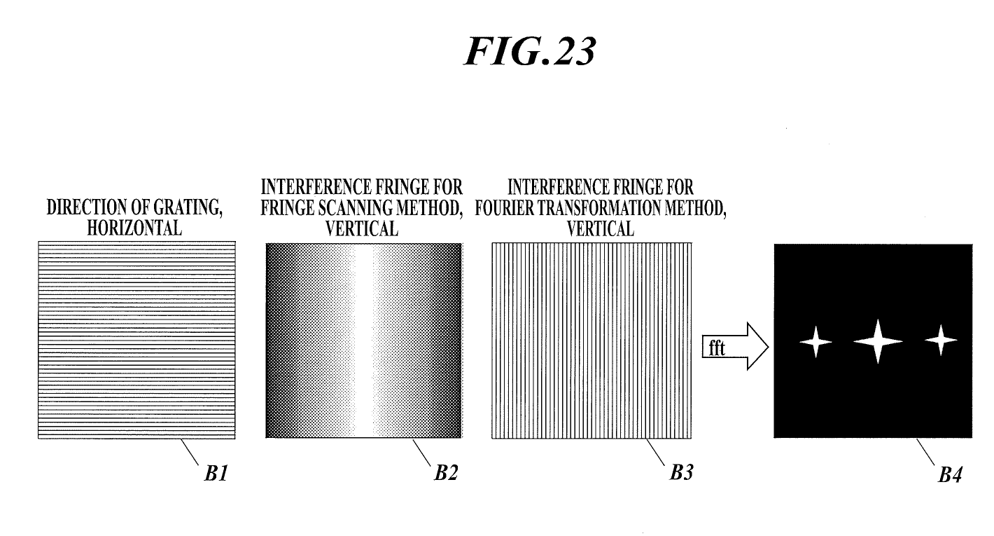

As shown in B1 of FIG. 23, when the directions of the gratings of the multi-slit 12, the first grating 14, and the second grating 15 are horizontal, the fringe of the moire image (an image which can be obtained by slightly tilting the second grating 15 with respect to the first grating 14) for the fringe scanning method becomes vertical as shown in B2 of FIG. 23. As shown in B3 of FIG. 23, the fringe of the moire image for Fourier transformation (image obtained by further tilting the second grating) becomes a vertical fringe finer compared to B2 of FIG. 23. As shown in B4 of FIG. 23, the image where the Fourier transformation is performed on the moire image for Fourier transformation becomes an image with the zero order component and the first order component aligned horizontally.

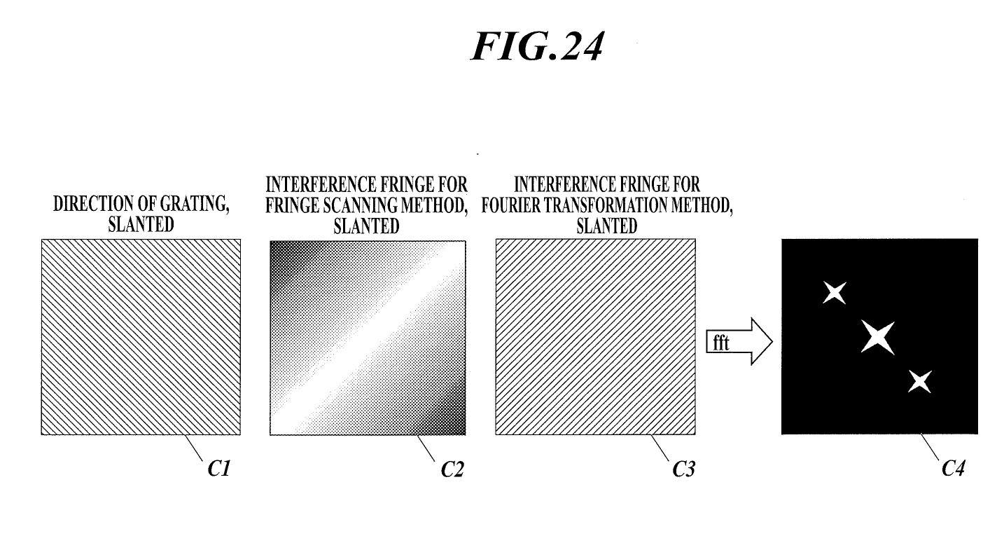

As shown in C1 of FIG. 24, when the directions of the multi-slit 12, the first grating 14, and the second grating 15 are slanted 45°, the fringe of the moire image (an image which can be obtained by slightly tilting the second grating 15 with respect to the first grating 14) for the fringe scanning method becomes slanted 45° (slanted in the opposite direction of the slit direction) as shown in C2 of FIG. 24. As shown in C3 of FIG. 24, the fringe of the moire image for Fourier transformation (image obtained by further tilting the second grating) becomes a slanted fringe in the same direction as C2 of FIG. 24 and finer than C2 of FIG. 24. As shown in C4 of FIG. 24, the image where the Fourier transformation is performed on the moire image for Fourier transformation becomes an image with the zero order component and the first order component aligned slanted 45° opposite of the fringe direction.

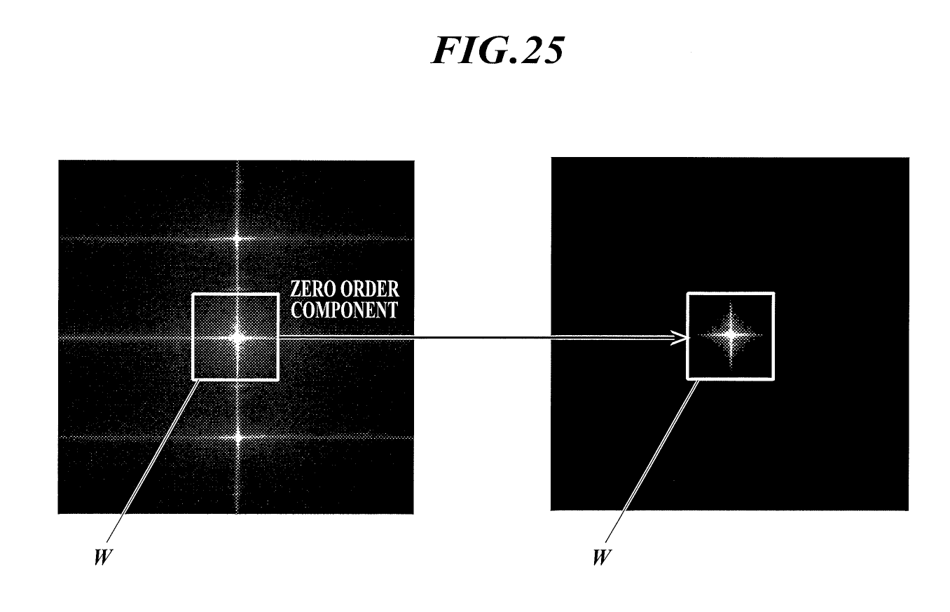

Next, in the image (with the subject and without the subject) obtained by the Fourier transformation, the zero order component is cut out by the Hanning window W shown in FIG. 25 (step S36). By cutting out with the Hanning window W, the surrounding section of the Hanning window W is dropped to 0 and the center section of the Hanning window W is passed as is.

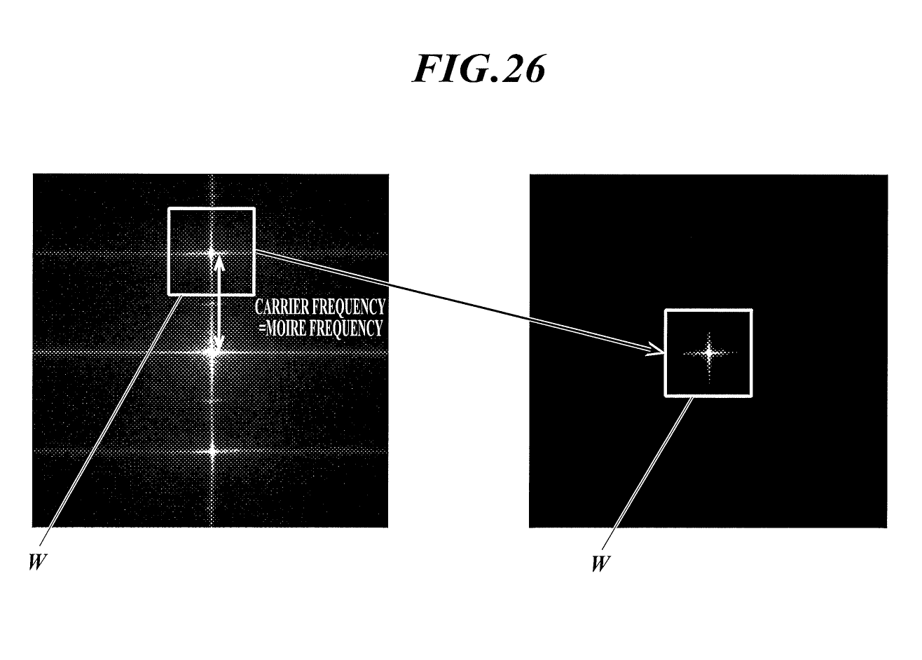

Next, in the image obtained by the Fourier transformation, the first order component is shifted in an amount of the carrier frequency (=moire frequency) as shown in FIG. 26, and cut out by the Hanning window W (step S37). The cut out window function is not limited to the Hanning window, and the Hamming window, the Gaussian window or the like may be used depending on the purpose.

Then, the inverse Fourier transformation is performed on the cut out zero order component and the first order component (step S38).

When the inverse Fourier transformation ends, each of the reconstructed image with the subject and without the subject is created using the zero order component and the first order component on which the inverse Fourier transformation is performed (step S39 to step S41). Specifically, the absorption image is created from the amplitude of the zero order component (step S39). Moreover, the differential phase image is created from the phase of the first order component (step S40). Moreover, the small angle scattering image is created from the ratio (=Visibility) of the amplitude between the zero order component and the first order component (step S41).

Next, the correction processing is performed in order to remove the phase of the interference fringe and to remove the image unevenness (artifact) from the reconstructed image with the subject using the reconstructed image without the subject (step S42). The processing of step S42 is similar to that described in step S21 of FIG. 14, and the description is incorporated herein.

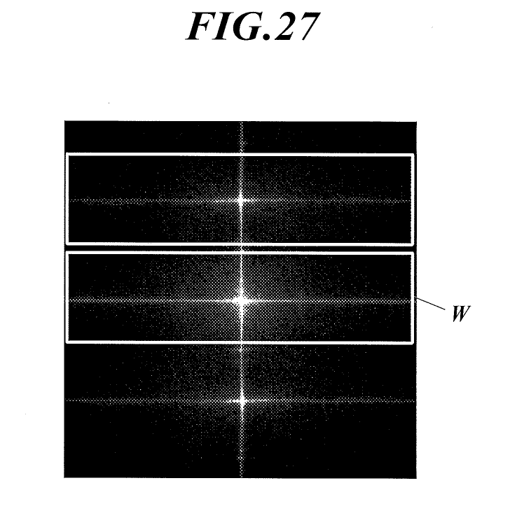

In the above described conventional Fourier transformation, when the zero order component and the first order component are cut out, the high frequency component is discarded vertically and horizontally. Therefore, the space resolution reduces and the entire image becomes blurred. Here, the inventors of the present application focuses on the point that, when one-dimensional grating is used as the X-ray grating, the information of the differential phase image and the small angle scattering image of the Talbot interferometer or the Talbot-Lau interferometer is only one direction which is the direction orthogonal to the slit direction of the grating (multi-slit 12, first grating 14, second grating 15). The inventors found that, instead of using a conventional square as the window W used in steps S36 and S37, by using a rectangle extending in a direction orthogonal to the slit direction of the grating as shown in FIG. 27, the components can be cut out without discarding the high frequency component of the signal in a direction orthogonal to the slit direction of the grating where image information is included. The inventors have also found that it is possible to reduce blurring in the direction orthogonal to the slit direction of the grating. (The above is called the improved Fourier transformation method). One of the features of the present invention is that the present invention uses the point that image information is initially not included in the direction parallel to the slit direction of the grating where reduction of the space resolution cannot be avoided in principle, and in the later described capturing performed twice, a large advantage can be achieved compared to the capturing by Fourier transformation method using a two-dimensional grating. (FIG. 27 shows an example of setting a rectangle window W in A4 of FIG. 22.)

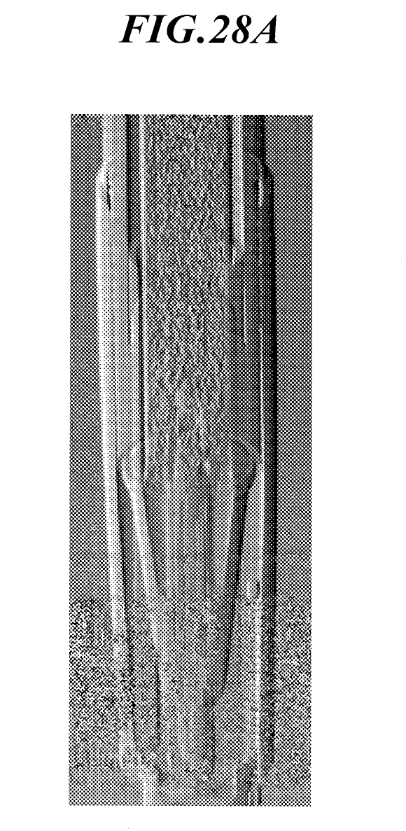

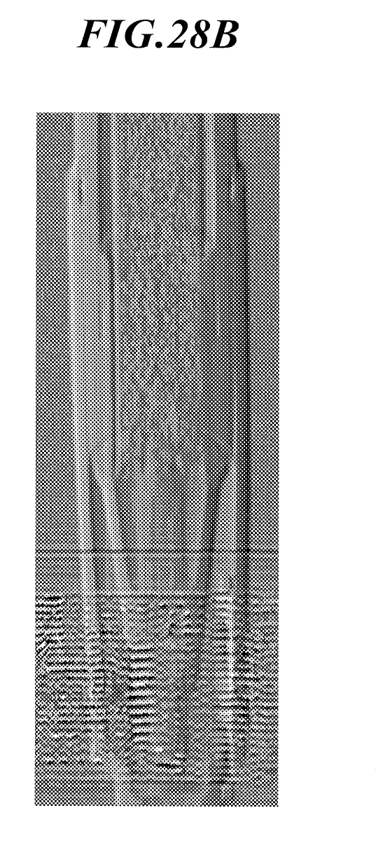

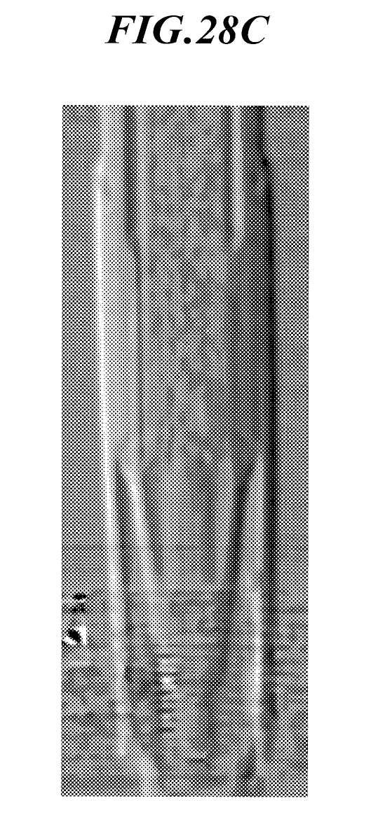

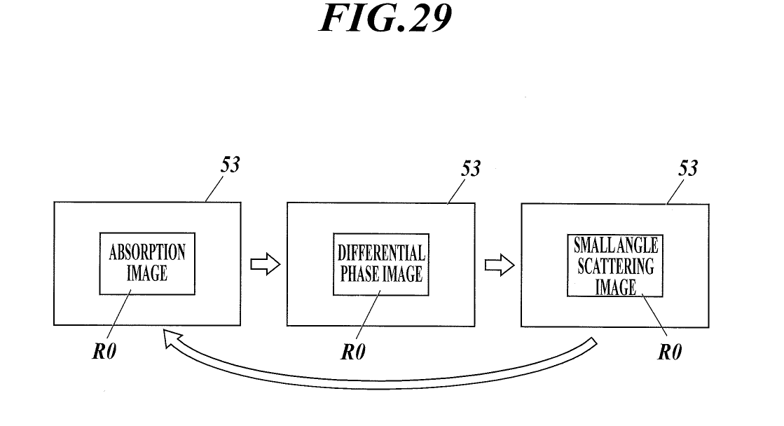

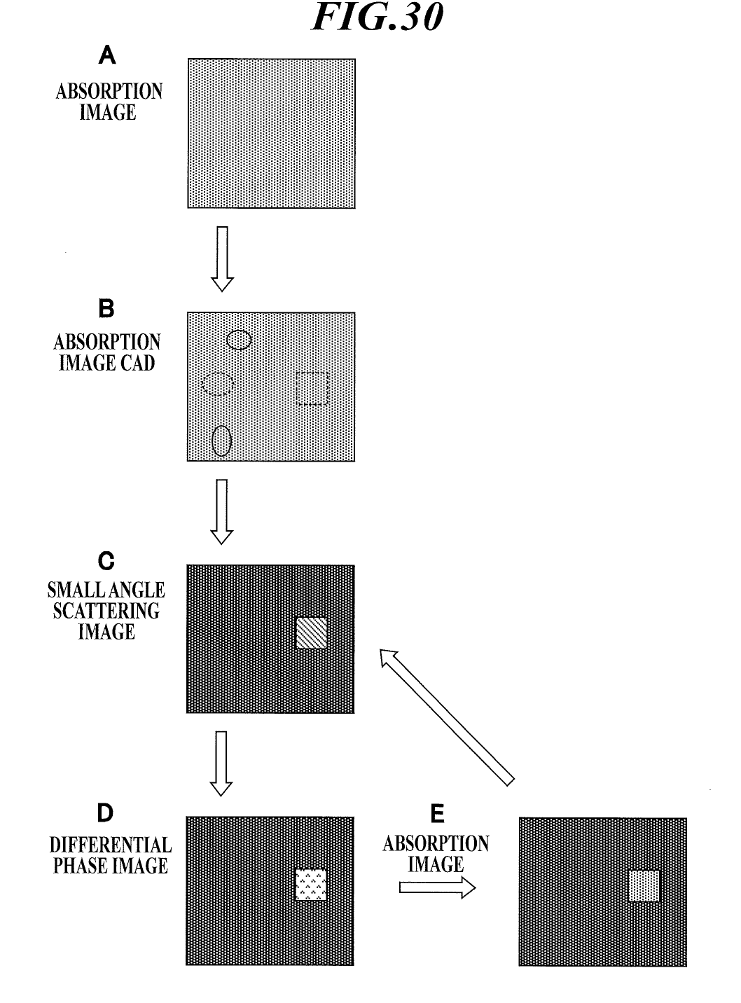

FIG. 28A shows an example of a reconstructed image of a subject obtained by the fringe scanning method. FIG. 28B shows an example of a reconstructed image obtained by the improved Fourier transformation method. FIG. 28C shows an example of a reconstructed image obtained by the conventional Fourier transformation method. The reconstructed images of FIG. 28A to FIG. 28C are differential phase images obtained by capturing with the slit direction of the grating in the vertical direction. As shown in FIG. 28A, the image obtained by the fringe scanning method is an image where the blurring in the vertical direction and the horizontal direction is small. As shown in FIG. 28B, the image obtained by the improved Fourier transformation is an image where only the vertical direction is blurred and the horizontal direction is not blurred. As shown in FIG. 28C, the image obtained by the conventional Fourier transformation method is an image blurred in both the vertical direction and the horizontal direction.

The differential phase image is shown in FIG. 28A to FIG. 28C, however, the direction of blurring seen in each method is similar in the absorption image and the small angle scattering image.

As described above, according to the improved Fourier transformation method, only the signal component in the direction parallel to the slit direction of the grating blurs. Therefore, it is possible to obtain a two-dimensional image with a small amount of blurring in both the vertical direction and the horizontal direction of the subject by the following method (in cases of differential phase image and small angle scattering image). The longitudinal direction of the subject is positioned in a direction orthogonal to the slit direction of the grating to perform the first capturing, and then the relative angle between the subject and the grating is rotated 90° to perform the second capturing. Then, reconstructed images are generated from the moire images obtained from the first and second capturing, and the two generated reconstructed images are combined.

Although it is possible to capture with the Fourier transformation method using the two-dimensional grating, since there is a first order element both vertically and horizontally, the range of the cut out window w is limited to a small range both vertically and horizontally. Therefore, it is not possible to avoid the space resolution drastically reducing. Turning to the present method, it is possible to generate the two-dimensional image without largely reducing the resolution by using the one-dimensional grating. In the combined image based on the capturing in two directions, the subject information in the four corners of the combined image is lost. However, in the radiation capturing in medical practice, typically the region of interest of the subject is mostly positioned in the center section of the capturing region. Therefore, such loss of the subject information hardly becomes a problem. Moreover, since the capturing itself is performed only two times, it is possible to reduce influence of the body movement of the subject.

When the capturing direction is changed (the slit direction of the subject is changed), similar to the fringe scanning method, the multi-slit 12, the first grating 14 and the second grating 15 need to be rotated 90° at the same time.

When the relative angle between the first grating 14 and the second grating 15 changes 90° and the first and the second capturing is performed, after the reconstructed image creating/displaying processing by the Fourier transformation method as shown in FIG. 19 is performed on the first captured image and the second captured image, the control section 51 of the controller 5 combines the two images. If the same portion of the subject is not drawn in the same pixel in the first image and the second image (if the subject is deformed or moved), either one of the images is moved parallel or rotated to be positioned where the error between the two images is smallest, and then the images are combined. As the method to combine the images, various methods can be employed. For example, the pixel of the first captured image is to be f1 (x, y), the pixel of the second captured image is to be f2 (x, y), the pixel of the combined image is to be g (x, y), and the following is calculated for each pixel to obtain the average value of the power (square root of the sum of squares).

g(x,y)=√(f1(x,y)̂2+f2(x,y)̂2)

For example, the captured images can be displayed in color, for example, the first captured image can be displayed in red and the second captured image can be displayed in blue.

As can be seen from the images of FIG. 28A to FIG. 28C, the reconstructed image obtained by the fringe scanning method is clearer and is less blurred than the reconstructed image obtained by the Fourier transformation. However, in the capturing for the fringe scanning method, since a plurality of images are captured successively, the capturing time becomes longer (about 1 minute) depending on the importing time of detector, the processing time before and after emitting the X-ray, mechanism operation time, etc., and the body tends to move. Turning to the Fourier transformation method, one image is captured in one session of capturing. Therefore, the capturing time depends on only the emitting time of the X-ray, and the time can be reduced to about 5 seconds. Consequently, it is expected that effect of reducing body movement can be achieved. Further, according to the improved Fourier transformation method, the degradation of the spatial resolution can be reduced. Therefore, by using both methods such as, (1) using the fringe scanning method when it is possible to fix the subject, and using the Fourier transformation method when it is desired to reduce the body movement, (2) using the Fourier transformation method in a simple examination, and using the fringe scanning method in a more accurate examination, it is possible to obtain the image suitable for a certain purpose and to perform capturing with less burden on the patient and less retries of capturing. According to the present embodiment, it is possible to easily switch between capturing for fringe scanning method and capturing for Fourier transformation method by easily adjusting the relative angle of the first grating 14 and the second grating 15. Therefore, it is possible to perform suitable capturing according to the purpose of the capturing.

Returning to FIG. 19, the created reconstructed image is displayed on the display section 53 (step S43).

Below, the display mode of the reconstructed image in step S22 of FIG. 14 and step S43 of FIG. 19 is described.

FIG. 29 shows an example of a display mode when the reconstructed image is displayed on the display section 53 in step S22 of FIG. 14 and step S43 of FIG. 19. As shown in FIG. 29, in step S22 and step S43, the three reconstructed images of the absorption image, the differential phase image, and the small angle scattering image are displayed in the same position (region R0) of the display section 53 sequentially switching and circulating the images each time a predetermined amount of time passes. Preferably, a stop button and a pause button are provided on the screen outside the image region of the display section 53, so that it is possible to continue displaying a certain image at a still state according to operation of the operation section 52.

The three reconstructed images of the absorption image, the differential phase image, and the small angle scattering image are created by performing different processing on the captured image (moire image) obtained in one capturing set. Therefore, as shown in FIG. 18A to FIG. 18C, the position of the subject in the three images are the same, and the images each show information of a different feature of the subject. For example, the absorption image shows information of a change in the large configuration. The differential phase image shows information of the phase change of the surrounding tissue. The small angle scattering image shows information of scattering in the tissue.

Therefore, as shown in FIG. 29, by displaying the three reconstructed images in the same position (region R0) on the display section 53 sequentially switching the images each time a predetermined amount of time passes, the physician who interprets the images does not have to move his line of sight and does not become tired. Therefore, the physician can interpret the images maintaining high degree of concentration. Moreover, due to the effect of the afterimage (subliminal effect) when the image is switched each time a predetermined amount of time passes, it is possible to reconstruct the plurality of pieces of information (features) regarding the subject in the physician's mind, and it is possible to perform diagnosis with high accuracy.

In FIG. 29, the three reconstructed images are displayed sequentially switched and circulated in the order of absorption image→differential phase image→small angle scattering image. However, the number of displayed images may be two or more. The order of switching display and the type of image is not limited, and may be set according to the site, or according to the user.

For example, when the subject site is a breast, it is preferable to switch the display in the order of absorption image→small angle scattering image→differential phase image so that the physician is able to perform diagnosis efficiently without spending excess time.

As described above, the information of the change in the large configuration appears in the absorption image. Therefore, by displaying the absorption image first, the physician is able to acknowledge the distribution of the fat and mammary gland in the entire breast and large lesions. Next, by displaying the small angle scattering image, the physician is able to detect with high sensitivity a portion accumulated with cancer tissue with calcification and without calcification, and is able to distinguish whether or not there is breast cancer. Next, by displaying the differential phase image, the edge section between the tumor or breast cancer tissue and the normal tissue can be detected, and it is possible to discriminate the range where the breast cancer spreads.

After the absorption image, it is possible to first display the differential phase image to detect the edge section between the tumor or the breast cancer tissue and the normal tissue to discriminate the range where the breast cancer spreads, and then to display the small angle scattering image to detect the portion accumulated with cancer tissue with calcification and without calcification. The order of which is displayed first may be determined according to each physician so that as a result, the abnormal shadow detectability of the physician is enhanced.

Moreover, the absorption image is conventionally used as a breast image for diagnosis and is an image that the physician is most used to (familiar) in diagnosis. Therefore, it is preferable to perform primary diagnosis based on diagnostic resolution which each physician acquired over the years of diagnosis. By displaying the absorption image, and then the small angle scattering image and the differential phase image, and performing interpretation again based on these images, it is possible to self-correct the primary diagnosis result of whether or not there is an abnormal shadow, whether the abnormal shadow is benign or malignant, etc. Moreover, by interpreting the images of the small angle scattering image and the differential phase image and then interpreting the absorption image again, it is possible to gradually confirm by sight the abnormal shadow, difference between benign and malignant, or the like which could not be seen at first. Therefore, by repeating the above diagnosis method, the physician is able to establish new diagnostic resolution at a higher level, and is able to perform diagnosis more accurate than before even in diagnosis based on only the absorption image, which is preferable.

Alternatively, when the subject site is the arms and legs, it is preferable to switch display in the order of absorption image→differential phase image. By displaying the absorption image first, the physician is able to guess where a cartilage or a tendon is, and then by displaying the differential phase image, the physician is able to discriminate whether or not the cartilage is worn or the tendon is ruptured.

### Second Embodiment

Below, the second embodiment of the present invention is described.

Conventionally, in order to support image diagnosis, an abnormal shadow candidate detecting apparatus (CAD: Computer-Aided Diagnosis) detects an abnormal shadow candidate from a medical image and the result of detecting is displayed with the medical image for diagnosis. Conventionally, as described above, an absorption image is used as the medical image for diagnosis, and only the absorption image is used when the abnormal shadow candidate is detected with the CAD.

In view of the above, as the second embodiment, described below is an example where, similar to the conventional absorption image based diagnosis system, first the interpreting of the image and the detecting of the abnormal shadow candidate with the CAD is performed with the absorption image among the reconstructed images, and then the small angle scattering image and the differential phase image are used in the secondary diagnosis to judge true positive/false positive of the region, etc. detected as the abnormal shadow candidate.

Regarding the medical image display system of the second embodiment, the configuration of the X-ray capturing apparatus 1 and the controller 5, and the operation from capturing to creating the reconstructed image are the same as those described in the first embodiment, therefore the description is incorporated herein.

In the controller 5, when the creating of the reconstructed image ends, the control section 51 performs the following processing in coordination with the program stored in the storage section 55.

First, as shown in A in FIG. 30, the absorption image among the reconstructed images is displayed on the display section 53. The physician observes and interprets the displayed absorption image. In A in FIG. 30, although drawing of the image is omitted, it is assumed that an image of a breast is drawn.

Next, according to an instruction from the operation section 52, the abnormal shadow candidate detecting processing is performed on the absorption image and the abnormal shadow candidate is detected from the absorption image. Here, in the second embodiment, the abnormal shadow candidate detecting program is stored in the storage section 55 of the controller 5, and the control section 51 of the controller 5 performs the abnormal shadow candidate detecting processing on the absorption image in coordination with the abnormal shadow candidate detecting program stored in the storage section 55.

As the detecting algorithm of the abnormal shadow candidate, a well known method can be applied. For example, as the algorithm of the tumor shadow candidate in the breast image, a method using the Iris filter disclosed in Japanese Unexamined Patent Application Publication No. H10-91758, a method using the Laplacian filter (Transactions of Institute of Electronics Information and Communication Engineers (D-II), Vol. J76-D-II, no. 2, pp. 241-249, 1993) and the like can be applied. As the detecting algorithm of the micro-calcification cluster shadow candidate, for example, a method using a morphology filter (Transactions of Institute of Electronics Information and Communication Engineers (D-II), Vol. J71-D-II, no. 7, pp. 1170-1176, 1992), a method using the Laplacian filter (Transactions of Institute of Electronics Information and Communication Engineers (D-II), Vol. J71-D-II, no. 10, pp. 1994-2001, 1998), a method using a triple ring filter, and the like can be applied.

When the detecting of the abnormal shadow candidate ends, as shown in B in FIG. 30, an annotation is displayed on the absorption image displayed on the display section 53, the annotation showing the position where the abnormal shadow candidate is detected. In B in FIG. 30, the elliptic annotation shows the position of the micro-calcification cluster shadow candidate and the rectangular annotation shows the position of the tumor shadow candidate. The annotation of the broken line shows a candidate with the possibility of being false positive.

Next, the physician refers to the medical image and the annotation displayed on the display section 53 and specifies a region of the candidate to be the target of secondary diagnosis with the operation section 52 (for example, double clicking). When the region of the candidate to be the target of the secondary diagnosis is specified with the operation section 52, display is performed according to the type of specified abnormal shadow candidate (for example, tumor, micro-calcification cluster, etc.).

For example, there is a possibility that a tumor may appear in any of the absorption image, the small angle scattering image, and the differential phase image. Therefore, when the rectangular region, in other words, the tumor candidate region is specified by the operation section 52, as shown in C to E in FIG. 30, the images are displayed switched and circulated each time a predetermined amount of time passes in the order of small angle scattering image→differential phase image→absorption image.

Specifically, first the small angle scattering image is displayed on the display section 53. Here, as shown in C in FIG. 30, in order to prevent misleading by unnecessary portions other than the specified region, the region other than the specified tumor candidate is processed to be blackened (state converted to black with low luminance) and then displayed. Next, after a predetermined amount of time passes, the display of the image is switched to the differential phase image. Similarly, as shown in D in FIG. 30, when the differential phase image is displayed, the region other than the specified region is processed to be blackened and then displayed. Next, after a predetermined amount of time passes, as shown in E in FIG. 30, the process displays the absorption image on which blackening processing is performed on the regions other than the specified region. The above is repeated.

For example, the micro-calcification cluster easily appears on the differential phase image. When the ellipse region, in other words, the region of the micro-calcification cluster candidate is specified by the operation section 52, the differential phase image→small angle scattering image→absorption image are displayed switched and circulated each time a predetermined amount of time passes. Here, in order to prevent misleading by unnecessary portions other than the specified region, the region other than the specified micro-calcification candidate is processed to be blackened and then displayed.

As described above, according to the present embodiment, it is possible to display the small angle scattering image and the differential phase image on the region of the abnormal shadow candidate detected by the CAD. Therefore, it is possible to judge the detected result of true positive/false positive of the abnormal shadow candidate based on the absorption image according to the small angle scattering image and/or the differential phase image which reproduces the features which do not appear on the absorption image of the subject.

In the conventional diagnosis system based on the absorption image, first the abnormal shadow candidate is detected on the absorption image obtained from the X-ray breast capturing in advance. When there is an abnormality, additional capturing is performed by other modalities such as an ultrasonic diagnostic device to enhance diagnosis accuracy. Then finally, diagnosis continues to a biopsy. Therefore, the patient needs to go to the hospital two times, which is a burden to the patient. There are cases where ultrasonic diagnosis is performed together, however, this becomes a waste to the patient if no abnormal shadow candidates are found. Further, the corresponding between the region of the abnormal shadow candidate detected with the CAD and the region captured with the ultrasonic diagnostic device, etc. depends on the operation by the operator. Therefore, there is a possibility that the error in corresponding leads to error in diagnosis.

According to the method of the present embodiment, the differential phase image and the small angle scattering image which are obtained in the same set of capturing as the absorption image used in detecting with the CAD and which show the features different from the absorption image are provided for the region in which the abnormal shadow candidate is detected with the CAD. Therefore, it is not necessary to perform capturing again for diagnosis such as ultrasonic diagnosis, etc., and the burden of the patient can be reduced. Moreover, it is possible to perform early diagnosis by the same physician. Also, since the subject and its position is the same as the absorption image, matching of the positions between the images of the two diagnoses do not have to be performed, and the positions are accurate. Therefore, it is possible to enhance diagnosis accuracy. Further, matching of the position is not performed and only blackening processing is performed. Therefore, it is possible to reduce processing time. Since the region other than the target of the secondary diagnosis is displayed with blackened processing, not much time is necessary for interpretation, and it is possible to enhance diagnosis performance.

Moreover, after the absorption image is displayed, the small angle scattering image and the differential phase image are displayed, and the interpretation of the images are performed again based on these images. Therefore, it is possible to self-correct the result of the primary diagnosis such as whether or not there is an abnormal shadow, benign/malignant of the abnormal shadow, or the like. Moreover, when the interpretation of the absorption image is performed again after interpreting the small angle scattering image and the differential phase image, the abnormal shadow, the difference of benign/malignant, or the like which could not be seen at first can be eventually confirmed by sight. By repeating the above diagnosis method, the physician is eventually able to establish new diagnostic resolution at a higher level, and is able to perform diagnosis more accurate than before even in diagnosis based on only the absorption image, which is preferable.

Which type of image among the small angle scattering image, differential phase image, and absorption image is switched and displayed at what timing can be set in advance with the operation section 52 for each user or for each site. Whether to perform blackened processing or to display the entire image region can also be set in advance.

Alternatively, first the abnormal shadow candidate can be detected in the absorption image, and then the small angle scattering image and the differential phase image can be created for only the detected region. With this, it is possible to further reduce the processing time.

The processing and display of the modified example as described below can be employed in the embodiment which uses the result of detecting the abnormal shadow candidate from the absorption image.

First, in the controller 5, when the creating of the reconstructed image ends, the control section 51 performs the abnormal shadow candidate detecting processing on the absorption image in coordination with the program stored in the storage section 55, and the abnormal shadow candidate is detected from the absorption image.

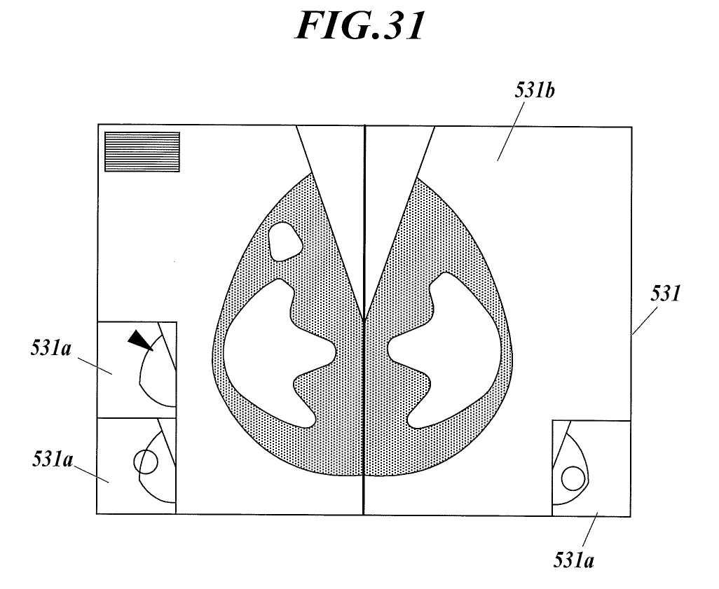

When the abnormal shadow candidate is detected, a reduced image 531a is created with annotation in the position of the abnormal shadow candidate detected on the absorption image. A left breast image and a right breast image of the small angle scattering image or the differential phase image are matched at the breast wall to create the main image 531b. Then, as shown in FIG. 31, the diagnosis screen 531 is displayed on the display section 53 with the main image 531b positioned in the center and the reduced image 531a positioned outside the subject region of the main image 531b.

In the modified example, similar to the second embodiment, the abnormal shadow candidate is detected on the absorption image as in conventional methods, and the result is displayed in the reduced image 531a in a position which does not interfere with the observation of the life size main image 531b including the differential phase image or the small angle scattering image. Therefore, it is possible to perform secondary diagnosis of whether the abnormal shadow candidate is true positive or false positive by observing the small angle scattering image or the differential phase image reproducing the features different from the absorption image while confirming the position of the abnormal shadow candidate detected based on the absorption image similar to the conventional method.

In the modified example, similar to the second embodiment, the image which is processed and created by the image obtained in the capturing at the same session as the absorption image used in the detecting by the CAD and which shows the features different from the absorption image is provided, therefore, the patient does not have to go to the hospital twice and the burden of the patient can be reduced. Moreover, early diagnosis by the same physician is possible. Moreover, it is possible to confirm the position of the abnormal shadow candidate with the reduced image in which the position of the subject is the same as the main image. Therefore, it is possible to accurately find the position of the abnormal shadow candidate from the main image to perform diagnosis.

Which type of image between the small angle scattering image or the differential phase image is displayed as the main image 531b can be set in advance with the operation section 52 for each user or each site. As the main image 531b, it is possible to display the images by switching between the small angle scattering image and the differential phase image. In this case, the processing uses the small angle scattering image and the differential phase image which are created by the image obtained from one capturing set and in which the position of the subject with respect to the detector is the same. Therefore, if the left breast image and the right breast image are matched at the position of the breast wall in the first image, the position matching processing does not need to be performed in the other image. Therefore, it is possible to provide the main image 531b in which the positions of the left and right breasts match by simply switching between the image data.

### Third Embodiment

Below, the third embodiment of the present invention is described.

In the third embodiment described below, the order of switching display as described in the first embodiment is determined using the result of detecting with the CAD.

Regarding the medical image display system of the third embodiment, the configuration of the X-ray capturing apparatus 1 and the controller 5, and the operation from capturing to creating the reconstructed image are the same as those described in the first embodiment, therefore the description is incorporated herein.

In the controller 5, when the creating of the reconstructed image ends, in coordination with the abnormal shadow candidate detecting program stored in the storage section 55, the control section 51 detects the abnormal shadow candidate from each of the absorption image, the small angle scattering image, and the differential phase image. The algorithm of the abnormal shadow candidate detecting program applied to each image is the same. After detecting, the image in which the abnormal shadow candidate is detected is set as the first image to be displayed, and as shown in FIG. 29, the absorption image, the small angle scanning image, and the differential phase image are displayed on the display section 53 switched and circulated each time a predetermined amount of time passes.

Specifically, the following cases are assumed as the examples of the result of detecting the abnormal shadow candidate in the absorption image, the small angle scattering image, and the differential phase image, and the order of displaying each image.

(1) Case where Abnormal Shadow Candidate is Detected from Only One Image

In this case, the images are displayed in the order of image in which the abnormal shadow candidate is detected→absorption image→remaining image. When the abnormal shadow candidate is detected from only the absorption image, first, the absorption image which the physician is familiar with is displayed. The order of priority between the small angle scattering image and the differential phase image can be set in advance from the operation section 52.

(2) Case where the Result of Detecting the Abnormal Shadow Candidate is the Same in Three Images

In this case, first, the absorption image which the physician is familiar with is displayed, and then, the other images are displayed according to the instruction to switch from the operation section 52. The type of image displayed according to the instruction to switch can be set in advance from the operation section 52. Alternatively, a display order where the physician can easily perform diagnosis can be set in advance.

(2a) Case where Abnormal Shadow Candidate is not Detected from all Three Images

The threshold used in detecting the abnormal shadow candidate in the abnormal shadow candidate detecting program is lowered, the abnormal shadow candidate detecting processing is performed again on each of the three images to detect the candidate closer to false positive, and similar to (1), the images are displayed in order from the image in which the abnormal shadow candidate is detected.

(2b) Case where Abnormal Shadow Candidate is Detected from all Three Images

The threshold used in detecting the abnormal shadow candidate in the abnormal shadow candidate detecting program is raised, the abnormal shadow candidate detecting processing is performed again on each of the three images to detect the candidate closer to true positive, and similar to (1), the images are displayed in order from the image in which the abnormal shadow candidate is detected.

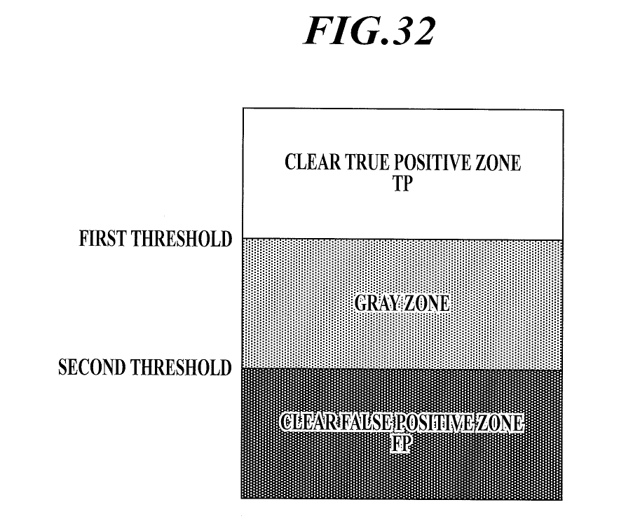

Here, the relationship between the abnormal shadow candidate and the threshold is described with reference to FIG. 32.

Typically, the abnormal shadow candidate detecting program primarily detects the candidate region with the possibility of being the abnormal shadow candidate using a predetermined detecting algorithm, and finally judges whether the detected primary candidate is the abnormal shadow candidate based on whether or not the feature amount (or the index value calculated from the feature amount) for each of the detected primary candidates exceeds a predetermined threshold (for example, see Japanese Unexamined Patent Application Publication No. 2007-151465). If it is considered that when the value exceeds the first threshold shown in FIG. 32, the value is in the clear true positive (TP) zone, and when the value is below the second threshold, the value is in the clear false positive (FP) zone. Then, the abnormal shadow candidate in between the first threshold and the second threshold is considered to be in the gray zone where the judgment of whether the result is true positive or false positive differs depending on the sensitivity of the abnormal shadow candidate detecting program. Regarding the candidate in the gray zone, it is necessary to judge separately whether or not there is an abnormality by a method other than using the CAD. Conventionally, the candidate in the gray zone is judged by other modalities. According to the above described (2a), when the abnormal shadow candidate is not detected in all of the images of the absorption image, the small angle scattering image, and the differential phase image, the threshold is lowered to be closer to the second threshold to be able to detect the abnormal shadow candidate in the gray zone closer to FP. Then, the physician first displays the image in which the abnormal shadow candidate is detected, so that the physician can concentrate on interpreting the image of the abnormal shadow candidate in the gray zone closer to TP. According to the above described (2b), when the abnormal shadow candidate is detected in all of the images of the absorption image, the small angle scattering image and the differential phase image, the threshold is raised to be closer to the first threshold to be able to detect the abnormal shadow candidate in the gray zone closer to TP. Then, by first displaying the image in which the abnormal shadow candidate is detected even if the threshold is raised, the physician can concentrate on interpreting the image of the abnormal shadow candidate in the gray zone closer to FP.

(3) Case where the Abnormal Shadow Candidate is not Detected from the Absorption Image but the Abnormal Shadow Candidate is Detected from the Differential Phase Image and the Small Angle Scattering Image

The image in which the detected number is larger and the absorption image are displayed switched between each other, on two screens at the same time, or overlapped in one screen.

(3a) Case where the Abnormal Shadow Candidate is not Detected from the Absorption Image but the Abnormal Shadow Candidate is Detected from the Differential Phase Image and the Small Angle Scattering Image

The threshold of the abnormal shadow candidate detecting program for the differential phase image and the small angle scattering image is raised, the abnormal shadow candidate detecting program is performed again, only the candidate closer to true positive is detected, and similar to (1), the image is displayed in order from the image in which the abnormal shadow candidate is detected.

In the above described third embodiment, the example described above applies the same abnormal shadow candidate detecting algorithm in the absorption image, the small angle scattering image and the differential phase image. However, it is possible to apply the abnormal shadow candidate detecting algorithm determined in advance for each image (including same algorithm with different threshold and different algorithm) and to display according to each case of (1) to (3a) as described above.

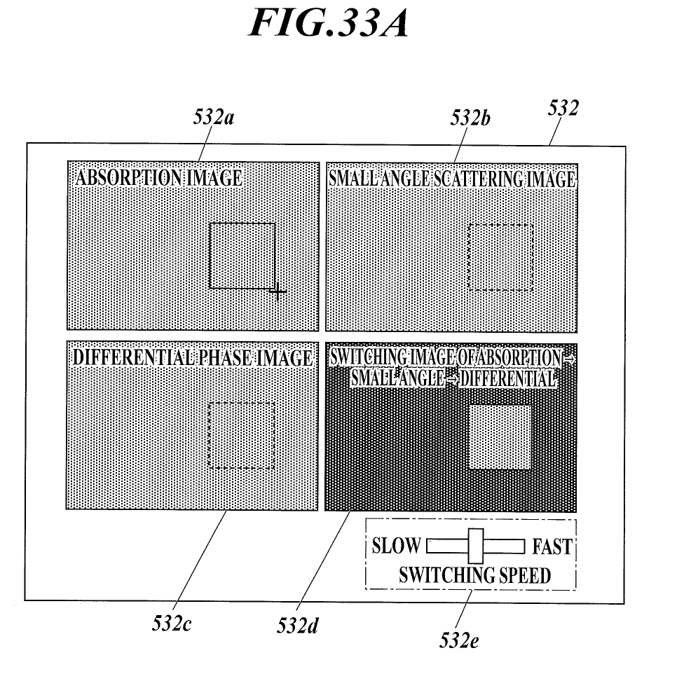

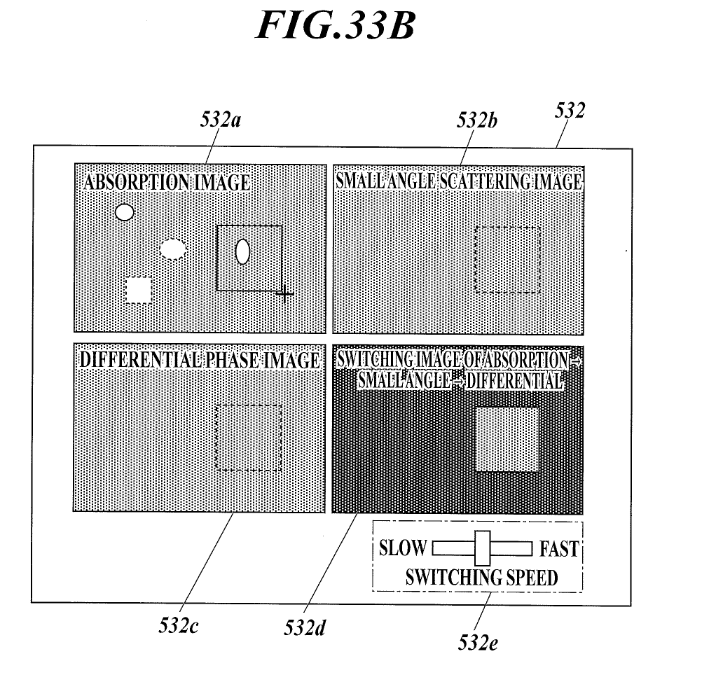

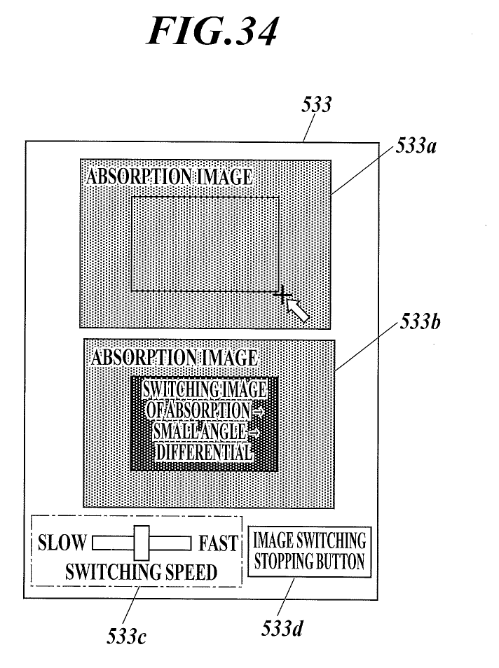

The display of switching among two or more types of images including the absorption image, the small angle scattering image, and the differential phase image may be performed alone as shown in FIG. 29 or the absorption image, the small angle scattering image, and the differential phase image may be displayed tiled on the screen of the display section 53 as shown in FIG. 33A, FIG. 33B, and FIG. 34.

FIG. 33A describes an example of a diagnosis screen 532 in which the absorption image 532a, the small angle scattering image 532b, the differential phase image 532c and the switching display image 532d are positioned tiled. The switching display image 532d is an image in which two or more types of images among the absorption image 532a, the small angle scattering image 532b, and the differential phase image 532c are displayed switched each time a predetermined amount of time passes.

In the diagnosis screen 532 shown in FIG. 33A, when an ROI is set on any of the images of the absorption image 532a, the small angle scattering image 532b, and the differential phase image 532c with the operation section 52, the ROI in the image is surrounded by a rectangle, etc. to be displayed identifiable, and as shown in FIG. 33A, the ROI is displayed identifiable in the other reconstructed images. Then, the region other than the ROI in the switching display image 532b is processed to be blackened. Moreover, as shown in FIG. 33A, a speed adjustment lever 532e is provided on the diagnosis screen 532, and the speed adjustment lever is operated with the operation section 52 so as to be able to adjust the interval of switching the images displayed on the switching display image 532d.

According to the diagnosis screen 532, for example, in the field of orthopedics of the arm, leg, etc., the physician observes the familiar absorption image 532a, and selects with the operation section 52 the region considered to be suspicious by inquiry. Then, together with the region selected in the absorption image, the corresponding region in the small angle scattering image and the differential phase image is displayed identifiable as the ROI. Therefore, it is possible to diagnose the wear and defect of the cartilage of the portion or diagnose whether there is a rupture in the tendon or the ligament with the small angle scattering image and the differential phase image. It is possible to perform combined diagnosis of information of each image with the display circulating, focusing on the position desired to be observed. According to the diagnosis screen 532, other than the display in which the images are switched, the three images showing the different features of the subject are displayed at the same time. Therefore, it is possible to finally confirm with the most characteristic image without any operation.

The abnormal shadow candidate may be detected from the absorption image with the CAD, and as shown in FIG. 33B, the annotation showing the position of the abnormal shadow candidate may be displayed in the absorption image 532a. Moreover, the region detected to be the abnormal shadow candidate by the CAD may be automatically set as the ROI.

As shown in the diagnosis screen 533 in FIG. 34, it is possible to display aligned in one screen the absorption image 533a, with which the physician is used to diagnosing, and the switching display image 533b. The switching display image 533b may display the absorption image in the initial display, and may switch to the display of the region set as the ROI in the absorption image 533a by the operation section 52.

The switching display image 532d, 533b can switch among the three images in the order of absorption image→small angle scattering image (differential phase image)→differential phase image (small angle scattering image), and as described above, the images can be switched according to the result of detecting the abnormal shadow candidate in the three images.

As described above, according to the medical image display system, the control section 51 of the controller 5 creates at least two of the X-ray absorption image, the differential phase image, and the small angle scattering image based on the moire image obtained by capturing in the first capturing mode by the fringe scanning type capturing apparatus and in the second capturing mode by the Fourier transformation type capturing apparatus in the X-ray capturing apparatus 1. Then, the display section 53 is controlled to display the created images on the display section 53.

For example, as described in the first embodiment, the control section 51 sequentially switches display of at least two images among the created X-ray absorption image, the differential phase image, and the small angle scattering image in the same position of the display section 53.

Specifically, when the subject site is the breast image, the display is controlled to sequentially switch display in the order of absorption image→small angle scattering image→differential phase image. The absorption image shows information of change of large configurations. Therefore, by displaying the absorption image first, the physician is able to acknowledge the distribution of fat and mammary gland and large lesions in the entire breast. Next, by displaying the small angle scattering image, it is possible to detect with high sensitivity the accumulating portion of cancer tissue with and without calcification and to determine whether or not there is breast cancer. Next, by displaying the differential phase image, it is possible to detect the edge section between the tumor or breast cancer tissue and the normal tissue, and it is possible to discriminate the range where the breast cancer spreads.

When the subject site is the arm, leg, etc., the display is controlled to switch in the order of absorption image→differential phase image. First the absorption image is displayed so that the physician is able to guess where a cartilage or tendon is, and then, the differential phase image is displayed so as to be able to judge whether or not there is wearing in the cartilage or rupture in the tendon.

As described above, by sequentially switching display in the same position of the display section 53 among at least two images of the X-ray absorption image, the differential phase image, and the small angle scattering image in the order according to the subject site, it is possible to effectively use the reconstructed image created from the moire image. With this, it is possible to realize early diagnosis and to enhance diagnosis accuracy.

As described in the third embodiment, the abnormal shadow candidate is detected with the CAD from each image among the X-ray absorption image, the differential phase image, and the small angle scattering image, and by controlling the type of image displayed and the order of the display when the images are displayed switched based on the detected result of each image, it is possible to display the images according to the detected result of the abnormal shadow candidate. Specifically, by displaying in order from the image in which the abnormal shadow candidate is detected, the physician can concentrate on interpreting the image of the portion detected as the abnormal shadow candidate, and thus it is possible to enhance diagnosis accuracy.

Moreover, as described in the third embodiment, by displaying each image of the X-ray absorption image, the differential phase image, and the small angle scattering image on the same screen as the switching display, the three images showing different features of the subject are displayed at the same time with the switching display. Therefore, after combined diagnosis of the features of each image with the switching display, it is possible to finally confirm diagnosis with the image showing the feature of the abnormal shadow candidate most.

As described in the second embodiment, for example, when the subject site is the breast, the control section 51 first displays the absorption image on the display section, then the abnormal shadow candidate is detected with the CAD from the absorption image similar to conventional methods, and when the detected abnormal shadow candidate is the tumor shadow candidate, the control section 51 controls display on the display section 53 switching the images in the order of small angle scattering image→differential phase image→absorption image. When the detected abnormal shadow candidate is the micro-calcified cluster, the control section 51 controls display on the display section 53 switching the images in the order of differential phase image→small angle scattering image→absorption image. Alternatively, the control section 51 controls display to display a diagnosis screen in which the differential phase image or the small angle scattering image is positioned in the center of the screen as the main image, and a reduced image displaying the position where the abnormal shadow candidate is detected on the absorption image is positioned outside the subject region of the main image.

As described above, regarding the region where the abnormal shadow candidate is detected with the CAD, by displaying on the display section 53 the differential phase image and the small angle scattering image which are obtained by the same capturing set as the absorption image used in detecting with the CAD and which show the features different from the absorption image, capturing again for ultrasonic diagnosis, etc., is not necessary. Consequently, it is possible to reduce the burden of the patient. Moreover, it is possible to achieve early diagnosis by the same physician. Moreover, since the subject and its position is the same as the absorption image, it is not necessary to match the positions between the images of the two diagnoses, and the positions are accurate. Consequently, it is possible to enhance diagnosis accuracy. Further, it is not necessary to match the position between images, and it is possible to reduce the processing time.

The above described embodiments are preferable examples of the present invention, and the embodiment is not limited to the above.

For example, according to the present embodiment described above, the X-ray capturing apparatus 1 is a configuration including a Talbot-Lau interferometer including a multi-slit, and the multi-slit is moved relatively with respect to the first grating and the second grating to generate a plurality of moire images for the fringe scanning method. However, the configuration may include a Talbot interferometer in which the first grating and the second grating are relatively moved in a certain cyclic interval, and the processing of the radiation detector reading the image signal in response to the X-ray emitted from the X-ray source for each movement in the certain cyclic interval can be repeated to generate a plurality of morie images for the fringe scanning method. Then, the plurality of moire images generated by the Talbot interferometer can be reconstructed to obtain the absorption image, the differential phase image, and the small angle scattering image, and the above images can be displayed sequentially switched in the same position of the display section 53 as described above.

The above described embodiment uses reconstructed images based on the one-dimensional image data captured by the apparatus which can capture in both the fringe scanning method and the Fourier transformation method (including the improved type). However, the present invention is not limited to the above and an apparatus dedicated to the Fourier transformation method (including the improved type) may be used.

Moreover, it is possible to apply the reconstructed image based on two-dimensional image data captured by a capturing apparatus dedicated to the Fourier transformation method in which the first grating and the second grating are two-dimensional grating or a capturing apparatus dedicated to the Fourier transformation method additionally using a multi grating (two-dimensional grating) near the focus position.

According to the present embodiment, the display method of the present invention is described with a case when the absorption image, the differential phase image, and the small angle scattering image are displayed sequentially switched in the same position of the display section 53. However, the display method of the present invention is not limited to the above, and the display method of the present invention can be applied to a case to display a plurality of images created by performing different image processing on the same captured image.

When the display is switched, it is possible to switch saturation (color) of each display screen, for example, black monotone, red monotone, or blue monotone, so that it is possible to easily confirm by sight that the image is switched.

According to the above embodiment, the components are positioned in the order of X-ray source 11, multi-slit 12, subject table 13, first grating 14, second grating 15, and X-ray detector 16 (hereinafter referred to as first position). However, even if the order is X-ray source 11, multi-slit 12, first grating 14, subject table 13, second grating 15, and X-ray detector 16 (hereinafter referred to as second position), the reconstructed image can be obtained by fixing the first grating 14 and the second grating 15, and moving the multi-slit 12.

According to the second position, the center of the subject and the first grating 14 are separated in the amount of the thickness of the subject, and the sensitivity is lower compared to the above embodiments. However, considering that the amount of radiation emitted to the subject is reduced, by employing the second position, it is possible to more effectively use the X-ray in the amount of X-ray absorbed by the first grating 14.

The effective spatial resolution in the subject position depends on the X-ray focus diameter, the spatial resolution of the detector, the magnification percentage of the subject, the thickness of the subject, etc. When the spatial resolution of the detector in the above embodiment is 120 μm (half width of gauss) or less, the effective spatial resolution becomes smaller in the second position than in the first position.

It is preferable to determine the order of the position of the first grating 14 and the subject table 13 considering sensitivity, spatial resolution, X-ray absorption amount by the first grating 14, and the like.

Moreover, the order of the capturing with the subject and the capturing without the subject is not limited to the above embodiment, and either one of the above can be performed first. The same can be said for the order of creating the reconstructed image with the subject and creating the reconstructed image without the subject.

Other than the above, the detailed configuration and the detailed operation of each apparatus composing the medical image display system can be suitably changed without leaving the scope of the invention.

The entire disclosure of Japanese Patent Application No. 2011-063470 filed on Mar. 23, 2011 including specification, claims, drawings and abstract are incorporated herein by reference in its entirety.

### INDUSTRIAL APPLICABILITY

As described above, the present invention can be used as a medical image display system which displays an X-ray image in the field of medicine.

### DESCRIPTION OF REFERENCE NUMERALS

1 X-ray capturing apparatus
11 X-ray source
12 multi-slit
12a rack
12b holder
121 multi-slit rotating section
121a motor section
121b gear section
121c gear section
121d supporting section
121e opening section
122 driving section
122a motor section
122b gear section
122c pinion
13 subject table
130 subject holder
131 ellipse shaped
133 interdigital spacer
14 first grating
140 grating section
141 first holder section
142 second holder section
142a protrusion section
15 second grating
150 grating section
151 holder section
16 X-ray detector
17 holding section
17a buffering member
171a opening section
171b tray fixing member
18 main section
181 control section
182 operation section
183 display section
184 communication section
185 storage section
18a driving section
210 grating rotating section
211 handle
212 rotating tray
212a opening section
212b to 212e depressed section
213 relative angle adjusting section
213a motor section
213b first gear
213c second gear
213d lever
214 stopper
5 controller
51 control section
52 operation section
53 display section
54 communication section
55 storage section

1. A medical image display system comprising:
a fringe scanning type capturing apparatus or a Fourier transformation type capturing apparatus including,
an X-ray source which emits an X-ray;
a first grating and a second grating;
a subject table; and
an X-ray detector including a conversion element provided two-dimensionally to generate an electric signal according to the emitted X-ray so that the X-ray detector reads the electric signal generated by the conversion element as an image signal;
an image processing section which generates a plurality of reconstructed images for diagnosis based on an image signal of a subject captured with either of the capturing apparatuses;
a display section which displays at least two of the plurality of reconstructed images for diagnosis generated by the image processing section; and
a control section which detects an abnormal candidate on each of the plurality of reconstructed images for diagnosis generated by the image processing section and which controls display order of the plurality of reconstructed images for diagnosis generated by the image processing section displayed on the display section based on a result of detecting.

2.-3. (canceled)

4. The medical image display system according to claim 1, wherein, the plurality of reconstructed images for diagnosis includes at least two among an absorption image, a differential phase image, and a small angle scattering image.

5. The medical image display system according to claim 1, wherein, the control section applies a same abnormal candidate detecting algorithm on the plurality of reconstructed images for diagnosis.

6. The medical image display system according to claim 1, wherein, the control section applies an abnormal candidate detecting algorithm on each of the plurality of reconstructed images for diagnosis predetermined for each of the plurality of reconstructed images for diagnosis.

7. A medical image display system comprising:
a fringe scanning type capturing apparatus or a Fourier transformation type capturing apparatus including, an X-ray source which emits an X-ray; a first grating and a second grating; a subject table; and an X-ray detector including a conversion element provided two-dimensionally to generate an electric signal according to the emitted X-ray so that the X-ray detector reads the electric signal generated by the conversion element as an image signal;
an image processing section which generates a plurality of reconstructed images for diagnosis based on an image signal of a subject captured with either of the capturing apparatuses;
a display section which displays at least two of the plurality of reconstructed images for diagnosis generated by the image processing section; and
a control section which controls a display order of the plurality of reconstructed images for diagnosis generated by the image processing section displayed on the display section according to a subject site.

8. The medical image display system according to claim 7, wherein, when the subject site is a breast, the control section displays the reconstructed images in a following order of an absorption image, a small angle scattering image, and a differential phase image.

9. The medical image display system according to claim 7, wherein, when the subject site is an arm or a leg, the control section displays the reconstructed images in a following order of an absorption image and a differential phase image.
